(19) United States
(12) Patent Application Publication
(10) Pub. No.: US 2004/0077879 A1
(43) Pub. Date: Apr. 22, 2004

(54) Bis (amino acid) molecular scaffolds

(76) Inventor: Christian E. Schafmeister, Pittsburgh, PA (US)

Correspondence Address: ECKERT SEAMANS CHERIN & MELLOTT, 600 GRANT STREET, 44TH FLOOR, PITTSBURGH, PA, 15219

(21) Appl. No.: 10/613,961

(22) Filed: Jul. 5, 2003

Related U.S. Application Data
(60) Provisional application No. 60/401,474, filed on Aug. 6, 2002.

(51) Int. Cl. C07D 487/02; C 07D 2 7/12

(52) U.S. Cl. 548/453; 548/537; 548/536

(57) ABSTRACT

The present invention provides molecular building blocks of rigid bis(amino acids). The molecular building blocks can be linked together through the formation of rigid diketopiperazine rings, to provide the desired three dimensional structure. Also provided is method of synthesizing macromolecules from the bis (amino acid) building blocks.

### CROSS-REFERENCE TO RELATED APPLICATIONS

This application claims priority to the Traditional Application entitled “BIS(AMINO ACID) MOLECULAR SCAFFOLDS”, filed Jul. 2, 2003 in the name of Christian E. Schafmeister and, under 35 USC 119(e), to provisional application Serial No. 60/401,474, filed Aug. 6, 2002, both expressly incorporated herein by reference.

### FIELD OF THE INVENTION

The present invention provides molecular building blocks of rigid bis(amino acids). The molecular building blocks can be linked together through the formation of rigid diketopiperazine rings, to provide the desired three dimensional structure.

### BACKGROUND INFORMATION

Biological proteins and catalytic RNA's are nature's general solution to the problem of how to construct nanoscale molecular devices. The powerful catalytic, information processing and energy transduction capabilities of proteins are examples of the powers inherent in molecules that are large enough to encapsulate smaller molecules and structured enough to position functional groups in three-dimensional space. Despite the thousands of high-resolution structures of biological proteins that have been determined and the several decades of effort from the computational biology community, the protein folding problem is still not well understood. Given an arbitrary primary sequence of a protein, the structure it will assume once it folds cannot be predicted. The converse problem, predicting the primary sequence of a protein that has a desired structure is also poorly understood. The de novo design of unnatural proteins is a very active area of research[1, 2] and has yielded several significant advances in the de novo design of simple α-helical and β-sheet proteins. But beyond very simple protein folds[3], the systematic construction of unnatural functional proteins is still not possible. The synthesis of poly-peptides both by chemical and biological means has become straight-forward, the harder problem is predicting how poly-peptides will fold.

Synthetic approaches to the synthesis of constitutionally precise macromolecules have been developed. Organic chemists are able to synthesize very large natural products, such as brevetoxin B [4-6]. However, as the size of a synthetic target increases, the amount of labor that is required to construct the molecule becomes prohibitive. Several groups have developed systematic approaches to the synthesis of constitutionally defined macromolecules such as Michl's “molecule sized Tinkertoy construction set” [7], Rebek's molecular capsules [8], Stoddart's “Molecular meccano kit” [9], and the supramolecular work of Jean-Marie Lehn [10], to name a few. These projects create molecules that are either flexible because they contain many freely rotating covalent bonds[7, 9] or they consist of supramolecular clusters held together by many weak non-covalent bonds[8-10]. Dendrimers and non-natural polymers are molecules with high molecular weights, but these molecules are also either highly flexible, highly symmetric or both [11]. Cyclophanes and cyclodextrins [12] are structurally well defined oligomers that attain a higher degree of structure than linear oligomers because they are constrained to form rings. These basket shaped molecules have generated a great deal of scientific interest as receptor and enzyme mimics but they are limited in the cavity sizes and shapes that they can form and in the complexity of the binding surface that they can present to guests.

New approaches to structured macromolecules such as the poly-β-peptide approach of Gellman and Seebaeh signify a tremendous advance [13-15]. These oligomeric molecules adopt complex secondary structure with as few as six residues. However, in order to create β-peptides with complex tertiary structure the folding rules of β-peptides will first need to be elucidated. The folding of β-peptides will rely on many weak non-covalent interactions to define tertiary structure. There is a need for molecular building blocks which can be linked together to create a pre-defined and desired shape.

### SUMMARY OF THE INVENTION

The present invention provides a new approach for synthesizing constitutionally precise macromolecules that will be used to design functional nanoscale molecular devices. The approach involves the coupling of synthetic stereochemically pure monomers through amide bonds to form oligomers in a manner similar to the synthesis of poly-peptides and poly-β-peptides. While traditional polypeptides and poly-β-peptides are assembled from flexible amino acids coupled through single amide bonds, the bis-peptides of the present invention are assembled from cyclic bis-amino acids that are coupled through pairs of amide bonds. This approach avoids the need to solve difficult folding problems because rigid ladder and spiro-ladder oligomers that contain no freely rotating bonds, within the core structure of the oligomer, are synthesized. Each monomer has a cyclic or fused ring structure and contains multiple stereocenters. Each monomer holds its two partners in a well defined orientation and distance with respect to each other. By assembling monomers in different sequences, an enormous number of macromolecules with different three-dimensional structures can be constructed (FIG. 2). These macromolecules can be used as scaffolds to present chemically reactive groups to carry out designed functions.

In contrast to previous methods, the present invention avoids the use of freely rotating covalent bonds in molecular scaffolds and reliance on the use of non-covalent interactions to maintain tertiary structure. This approach creates molecules that are highly structured and highly asymmetric, and can create cavities with an enormous variety of shapes and sizes while presenting a wide range of functional groups.

The molecular scaffolds can satisfy Cram's requirements of pre-organization and complementarity to act as hosts. An important requirement of macromolecules that will display biomimetic function is that they be capable of acting as hosts for small molecules. Pre-organization enhances binding by reducing the enthalphic cost of reorganization and the entropic cost of ordering the receptor on binding its guest. Molecular recognition occurs when the guest attaches non-covalently to the host, and is driven by a reduction of free energy associated with the formation of many complementary hydrophobic and electrostatic contacts between the host and guest. Each complementary contact contributes a very small amount of stabilization to the overall interaction. Every binding interaction must involve an interaction free energy of several times kT in order to at least overcome the loss of rotational and translational entropy of about 4.5-6.0 kcal/mole [16] when the complex forms. Many biological hosts almost completely encapsulate their guests because many weak complementary contacts must work together to create a strong interaction. A molecular host is an orderly array of functional groups, maintained in a pre-organized arrangement in three-dimensional space around a cavity [17]. In aqueous solution, functional groups include hydrophobic groups, hydrogen bonding groups, charged groups, aromatic groups and so on. In organic solvents, hydrogen bonding becomes very stabilizing and solvophobic effects are weaker. The molecular scaffolds of the present invention are highly ordered but complex macromolecules fully capable of displaying pre-organization as described by Cram [18]. They can also display cavities that contain several functional groups and thus should be fully capable of displaying the property of complementarity to desired guests.

This approach complements and offers advantages over other synthetic approaches to macromolecules. Many research groups have designed hosts and demonstrated binding to guests. Wilcox and co-workers have designed a minimalist host that displays two carboxylic acid groups and binds adenine and biotin derivatives strongly in organic solvents but weakly in methanol-water mixtures [19]. Diederich and co-workers have constructed a porphyrin-bridged cyclophane that binds arenes and catalyzes their oxidation with a turn-over number of about fourteen in methanol-d4/D2O/acetie acid (95:4.85:0.15% v/v) mixtures. Rebek and co-workers have made many host molecules including self-assembling capsules[20] that dimerize and bind Boc protected amino esters in mesitylene D₁₂ solvent. All of these functional molecules are assembled with short syntheses. The Rebek capsule is synthesized in two steps, the Wilcox molecule in four steps, and the Diederich molecule in about twelve steps. The building blocks of the present invention are synthesized in eight to fourteen steps, and will be assembled on solid support or in solution in a multi-step synthesis involving two steps for every building block.

The molecular scaffold methodology of the present invention is significant because it offers a systematic approach to the construction of macromolecules with precise control over size, shape, chemical and mechanical properties. This will have great future value in the design of nanoscale devices and macromolecules with biomimetic function.

It is an object of the present invention, therefore, to provide molecular building blocks which can be assembled into discrete shapes.

It is an additional object of the present invention to provide molecular building blocks made from bis (amino acids).

It is a further object of the present invention to provide a method of synthesis of bis peptides, using the bis (amino acid) molecular building blocks.

These and other objects will become more readily apparent from the following detailed description, drawings, and appended claims.

### BRIEF DESCRIPTION OF THE DRAWINGS

The invention is further illustrated by the following non-limited drawings in which:

FIG. 1: The general structures of nine classes of bis-amino acid molecular building blocks.

FIG. 2: A two-dimensional diagram illustrating the approach to macromolecule construction.

FIG. 3: The two phases of scaffold synthesis, elongation followed by rigidification.

FIG. 4: The four building blocks in the pro4 monomer class.

FIG. 5: The crude C₁₈ HPLC chromatogram of the five-mer (NH2-Tyr-1:pro4 (2S4S)5).

FIG. 6: An alphabet soup of predicted structures for headed arrows represent expected close contacts that will NMR spectra.

FIG. 7: The time dependant product formation of scaffolds containing “n” diketopiperazines.

FIG. 8: The four members of the pro3 monomer class.

FIG. 9: The four accessible stereoisomeric members of the pro4a monomer class. “R” is a suitable functional group.

FIG. 10: Various amines that could be coupled to 75 to create functionalized monomers.

FIG. 11: the reaction scheme for 1:pro4(2S4S)

FIG. 12: the reaction scheme for 2:pro4(2S4R)

FIG. 13: the reaction scheme for 3:pro4(2R4R) and 4:pro4(2R4S).

FIG. 14: the reaction scheme for synthesis of the three-mer scaffold 19.

FIG. 15: the reaction scheme for synthesis of the five-mer scaffold 33.

FIG. 16: the reaction scheme for synthesis of 44.

FIG. 17: the reaction scheme for synthesis of 35.

FIG. 18: the reaction scheme for synthesis of pro3(2R3S) and pro3(2R3R).

FIG. 19: reaction schemes for synthesis of the pip4 and pip5 classes.

FIG. 20: the reaction scheme for synthesis of the hin class.

FIG. 21: the reaction scheme for synthesis of 72 and 73.

FIG. 22: the reaction scheme for synthesis of 76.

FIG. 23: the reaction scheme for synthesis of pro4a(2S3R4S)NHR.

FIG. 24: the reaction scheme for synthesis of pro3r(2S3S).

FIG. 25: Sequence of three-mer 19.

FIG. 26: Sequence of compounds 21, 22, 23 and 24.

### DETAILED DESCRIPTION OF THE INVENTION

In one aspect, the present invention provides bis amino acids, that have been suitably modified to enable them to carry out the diketopiperization reaction, described more fully below, to form the rigid macromolecule of interest. As used herein, the term “bis amino acid” refers to any suitably modified amino acid, as exemplified by the structures below, that can provide the necessary reactivity for the diketopiperization reaction. Suitable bis amino acids include, but are not limited to, those structures specifically defined below.

In one embodiment, the present invention provides compounds having the formula

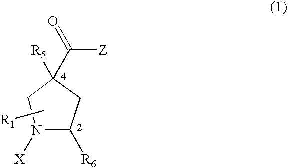

where:

X represents a first amine protecting group;

Y represents a second amine protecting group;

Z represents a weak leaving group;

R₁ represents an H, or a functional group, and can be attached to the molecule at positions 2, 3 or 5;

R₂ represents an H or a functional group;

R₅ represents N₃ or NR₂Y;

R₆ represents a carboxylic acid or a strongly activated ester; and

the stereochemical configuration at positions 2 and 4 and of the carbon bearing R₁ (if R₁ is not H) can be any one of (S,S,S), (S,S,R), (S,R,S), (S,R,R), (R,S,S), (R,S,R), (R,R,S) or (R,R,R).

In an additional embodiment, the present invention provides compounds having the formula

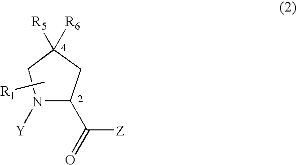

where:

X represents a first amine protecting group;

Y represents a second amine protecting group;

Z represents a weak leaving group;

R₁ represents an H, or a functional group, and can be attached to the molecule at positions 2, 3 or 5;

R₂ represents an H or a functional group;

R₅ represents N₃ or NR₂X;

R₆ represents a carboxylic acid or a strongly activated ester; and

the stereochemical configuration at positions 2 and 4 and of the carbon bearing R₁ (if R₁ is not H) can be any one of (S,S,S), (S,S,R), (S,R,S), (S,R,R), (R,S,S), (R,S,R), (R,R,S) or (R,R,R).

In an additional embodiment, the present invention provides compounds having the formula

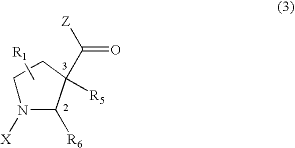

where:

X represents a first amine protecting group;

Y represents a second amine protecting group;

Z represents a weak leaving group;

R₁ represents an H, or a functional group, and can be attached to the molecule at positions 2, 4 or 5;

R₂ represents an H or a functional group;

R₅ represents N₃ or NR₂Y;

R₆ represents a carboxylic acid or a strongly activated ester; and

the stereochemical configuration at positions 2 and 3 and of the carbon bearing R₁ (if R₁ is not H) can be any one of (S,S,S), (S,S,R), (S,R,S), (S,R,R), (R,S,S), (R,S,R), (R,R,S) or (R,R,R).

In another embodiment, the present invention provides compounds having the formula

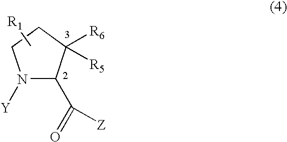

where:

X represents a first amine protecting group;

Y represents a second amine protecting group;

Z represents a weak leaving group;

R₁ represents an H, or a functional group, and can be attached to the molecule at positions 2, 4 or 5;

R₂ represents an H or a functional group;

R₅ represents N₃ or NR₂X;

R₆ represents a carboxylic acid or a strongly activated ester; and

the stereochemical configuration at positions 2 and 3 and of the carbon bearing R₁ (if R₁ is not H) can be any one of (S,S,S), (S,S,R), (S,R,S), (S,R,R), (R,S,S), (R,S,R), (R,R,S) or (R,R,R).

In an additional embodiment, the present invention provides compounds having the formula

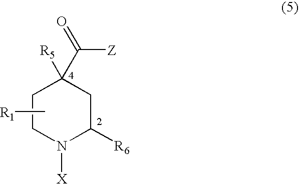

where:

X represents a first amine protecting group;

Y represents a second amine protecting group;

Z represents a weak leaving group;

R₁ represents an H, or a functional group, and can be attached to the molecule at positions 2, 3, 5 or 6;

R₂ represents an H or a functional group;

R₅ represents N₃ or NR₂Y;

R₆ represents a carboxylic acid or a strongly activated ester; and

the stereochemical configuration at positions 2 and 4 and of the carbon bearing R₁ (if R₁ is not H) can be any one of (S,S,S), (S,S,R), (S,R,S), (S,R,R), (R,S,S), (R,S,R), (R,R,S) or (R,R,R).

In an additional embodiment, the present invention provides compounds having the formula

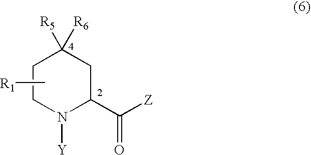

where:

X represents a first amine protecting group;

Y represents a second amine protecting group;

Z represents a weak leaving group;

R₁ represents an H, or a functional group, and can be attached to the molecule at positions 2, 3, 5 or 6;

R₂ represents an H or a functional group;

R₅ represents N₃ or NR₂X;

R₆ represents a carboxylic acid or a strongly activated ester; and

the stereochemical configuration at positions 2 and 4 and of the carbon bearing R₁ (if R₁ is not H) can be any one of (S,S,S), (S,S,R), (S,R,S), (S,R,R), (R,S,S), (R,S,R), (R,R,S) or (R,R,R).

In an additional embodiment, the present invention provides compounds having the formula

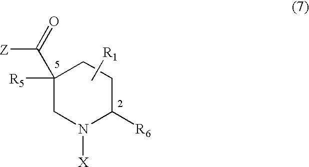

where:

X represents a first amine protecting group;

Y represents a second amine protecting group;

Z represents a weak leaving group;

R₁ represents an H, or a functional group, and can be attached to the molecule at positions 2, 3, 4 or 6;

R₂ represents an H or a functional group;

R₅ represents N₃ or NR₂Y;

R₆ represents a carboxylic acid or a strongly activated ester; and

the stereochemical configuration at positions 2 and 5 and of the carbon bearing R₁ (if R₁ is not H) can be any one of (S,S,S), (S,S,R), (S,R,S), (S,R,R), (R,S,S), (R,S,R), (R,R,S) or (R,R,R).

In an additional embodiment, the present invention provides compounds having the formula

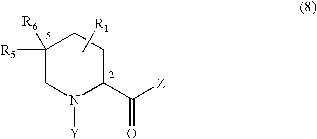

where:

X represents a first amine protecting group;

Y represents a second amine protecting group;

Z represents a weak leaving group;

R₁ represents an H, or a functional group, and can be attached to the molecule at positions 2, 3, 5 or 6;

R₂ represents an H or a functional group;

R₅ represents N₃ or NR₂X;

R₆ represents a carboxylic acid or a strongly activated ester; and

the stereochemical configuration at positions 2 and 5 and of the carbon bearing R₁ (if R₁ is not H) can be any one of (S,S,S), (S,S,R), (S,R,S), (S,R,R), (R,S,S), (R,S,R), (R,R,S) or (R,R,R).

In an additional embodiment, the present invention provides compounds having the formula

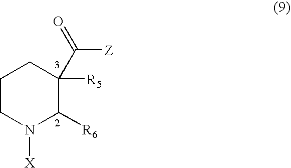

where:

X represents a first amine protecting group;

Y represents a second amine protecting group;

Z represents a weak leaving group;

R₁ represents an H, or a functional group, and can be attached to the molecule at positions 2, 4, 5 or 6;

R₂ represents an H or a functional group;

R₅ represents N₃ or NR₂Y;

R₆ represents a carboxylic acid or a strongly activated ester; and

the stereochemical configuration at positions 2 and 3 and of the carbon bearing R₁ (if R₁ is not H) can be any one of (S,S,S), (S,S,R), (S,R,S), (S,R,R), (R,S,S), (R,S,R), (R,R,S) or (R,R,R).

In an additional embodiment, the present invention provides compounds having the formula

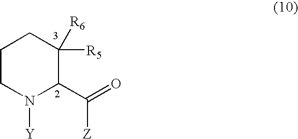

where:

X represents a first amine protecting group;

Y represents a second amine protecting group;

Z represents a weak leaving group;

R₁ represents an H, or a functional group, and can be attached to the molecule at positions 2, 4, 5 or 6;

R₂ represents an H or a functional group;

R₅ represents N₃ or NR₂X;

R₆ represents a carboxylic acid or a strongly activated ester; and

the stereochemical configuration at positions 2 and 3 and of the carbon bearing R₁ (if R₁ is not H) can be any one of (S,S,S), (S,S,R), (S,R,S), (S,R,R), (R,S,S), (R,S,R), (R,R,S) or (R,R,R).

In an additional embodiment, the present invention provides compounds having the formula

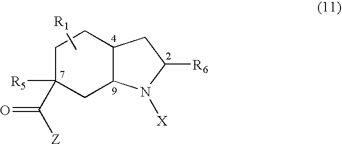

where:

X represents a first amine protecting group;

Y represents a second amine protecting group;

Z represents a weak leaving group;

R₁ represents an H, or a functional group, and can be attached to the molecule at positions 2, 3, 4, 5, 6, 8 or 9;

R₂ represents an H or a functional group;

R₅ represents N₃ or NR₂Y;

R₆ represents a carboxylic acid or a strongly activated ester; and

the stereochemical configuration at positions 2, 4, 7, 9 and of the carbon bearing R₁ (if R₁ is not H) can be any of the 32 combinations of (R) and (S).

In an additional embodiment, the present invention provides compounds having the formula

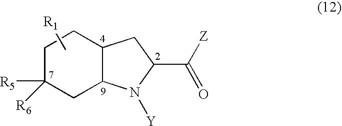

where:

X represents a first amine protecting group;

Y represents a second amine protecting group;

Z represents a weak leaving group;

R₁ represents an H, or a functional group, and can be attached to the molecule at positions 2, 3, 4, 5, 6, 8 or 9;

R₂ represents an H or a functional group;

R₅ represents N₃ or NR₂X;

R₆ represents a carboxylic acid or a strongly activated ester; and

the stereochemical configuration at positions 2, 4, 7, 9 and of the carbon bearing R₁ (if R₁ is not H) can be any of the 32 combinations of (R) and (S).

In an additional embodiment, the present invention provides compounds having the formula

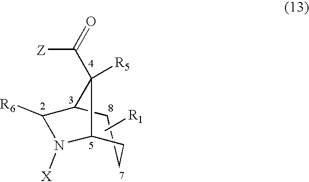

where:

X represents a first amine protecting group;

Y represents a second amine protecting group;

Z represents a weak leaving group;

R₁ represents an H, or a functional group, and can be attached to the molecule at positions 2, 3, 5, 6, 7 or 8;

R₂ represents an H or a functional group;

R₅ represents N₃ or NR₂Y;

R₆ represents a carboxylic acid or a strongly activated ester; and

the stereochemical configuration at the positions 2, 3, 4 and 5, and of the carbon bearing R₁ (if R₁ is not H) can be any of the 32 combinations of (R) and (S).

In an additional embodiment, the present invention provides compounds having the formula

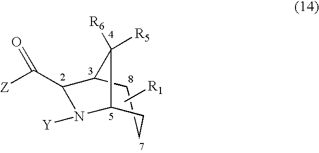

where:

X represents a first amine protecting group;

Y represents a second amine protecting group;

Z represents a weak leaving group;

R₁ represents an H, or a functional group, and can be attached to the molecule at positions 2, 3, 5, 6, 7 or 8;

R₂ represents an H or a functional group;

R₅ represents N₃ or NR₂X;

R₆ represents a carboxylic acid or a strongly activated ester; and

the stereochemical configuration at the positions 2, 3, 4 and 5, and of the carbon bearing R₁ (if R₁ is not H) can be any of the 32 combinations of (R) and (S).

In an additional embodiment, the present invention provides compounds having the formula

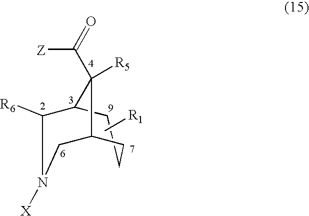

where:

X represents a first amine protecting group;

Y represents a second amine protecting group;

Z represents a weak leaving group;

R₁ represents an H, or a functional group, and can be attached to the molecule at positions 2, 3, 5, 6, 7, 8 or 9;

R₂ represents an H or a functional group;

R₅ represents N₃ or NR₂Y;

R₆ represents a carboxylic acid or a strongly activated ester; and

the stereochemical configuration at the positions 2, 3, 4 and 5, and of the carbon bearing R₁ (if R₁ is not H) can be any of the 32 combinations of (R) and (S).

In an additional embodiment, the present invention provides compounds having the formula

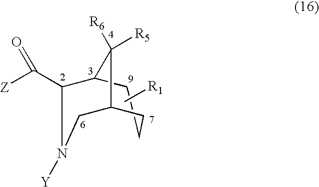

where:

X represents a first amine protecting group;

Y represents a second amine protecting group;

Z represents a weak leaving group;

R₁ represents an H, or a functional group, and can be attached to the molecule at positions 2, 3, 5, 6, 7, 8 or 9;

R₂ represents an H or a functional group;

R₅ represents N₃ or NR₂X;

R₆ represents a carboxylic acid or a strongly activated ester; and

the stereochemical configuration at the positions 2, 3, 4 and 5, and of the carbon bearing R₁ (if R₁ is not H) can be any of the 32 combinations of (R) and (S).

In an additional embodiment, the present invention provides compounds having the formula

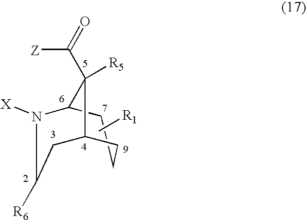

where:

X represents a first amine protecting group;

Y represents a second amine protecting group;

Z represents a weak leaving group;

R₁ represents an H, or a functional group, and can be attached to the molecule at positions 2, 3, 4, 6, 7, 8 or 9;

R₂ represents an H or a functional group;

R₅ represents N₃ or NR₂Y;

R₆ represents a carboxylic acid or a strongly activated ester; and

the stereochemical configuration at the positions 2, 4, 5 and 6, and of the carbon bearing R₁ (if R₁ is not H) can be any of the 32 combinations of (R) and (S).

In an additional embodiment, the present invention provides compounds having the formula

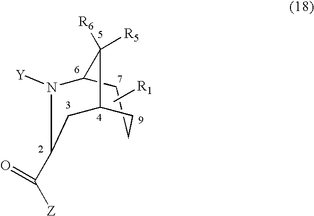

where:

X represents a first amine protecting group;

Y represents a second amine protecting group;

Z represents a weak leaving group;

R₁ represents an H, or a functional group, and can be attached to the molecule at positions 2, 3, 4, 6, 7, 8 or 9;

R₂ represents an H or a functional group;

R₅ represents N₃ or NR₂X;

R₆ represents a carboxylic acid or a strongly activated ester; and

the stereochemical configuration at the positions 2, 4, 5 and 6, and of the carbon bearing R₁ (if R₁ is not H) can be any of the 32 combinations of (R) and (S).

In an additional embodiment, the present invention provides compounds having the formula

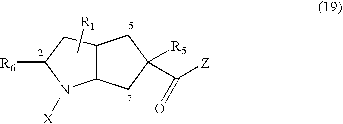

where:

X represents a first amine protecting group;

Y represents a second amine protecting group;

Z represents a weak leaving group;

R₁ represents an H, or a functional group, and can be attached to the molecule at positions 2, 3, 4, 5, 7 or 8;

R₂ represents an H or a functional group;

R₅ represents N₃ or NR₂Y;

R₆ represents a carboxylic acid or a strongly activated ester; and

the stereochemical configuration at the positions 2, 4, 6 and 8, and of the carbon bearing R₁ (if R₁ is not H) can be any of the 32 combinations of (R) and (S).

In an additional embodiment, the present invention provides compounds having the formula

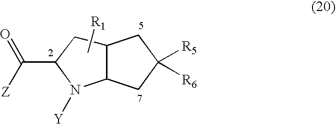

where:

X represents a first amine protecting group;

Y represents a second amine protecting group;

Z represents a weak leaving group;

R₁ represents an H, or a functional group, and can be attached to the molecule at positions 2, 3, 4, 5, 7 or 8;

R₂ represents an H or a functional group;

R₅ represents N₃ or NR₂X;

R₆ represents a carboxylic acid or a strongly activated ester; and

the stereochemical configuration at the positions 2, 4, 6 and 8, and of the carbon bearing R₁ (if R₁ is not H) can be any of the 32 combinations of (R) and (S).

In an additional embodiment, the present invention provides compounds having the formula

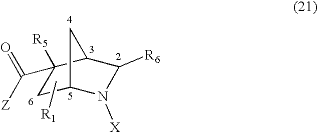

where:

X represents a first amine protecting group;

Y represents a second amine protecting group;

Z represents a weak leaving group;

R₁ represents an H, or a functional group, and can be attached to the molecule at positions 2, 3, 4, 5 or 6;

R₂ represents an H or a functional group;

R₅ represents N₃ or NR₂Y;

R₆ represents a carboxylic acid or a strongly activated ester; and

the stereochemical configuration at the positions 2, 3, 5 and 7, and of the carbon bearing R₁ (if R₁ is not H) can be any of the 32 combinations of (R) and (S).

In an additional embodiment, the present invention provides compounds having the formula

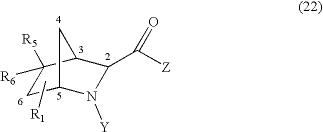

where:

X represents a first amine protecting group;

Y represents a second amine protecting group;

Z represents a weak leaving group;

R₁ represents an H, or a functional group, and can be attached to the molecule at positions 2, 3, 4, 5 or 6;

R₂ represents an H or a functional group;

R₅ represents N₃ or NR₂X;

R₆ represents a carboxylic acid or a strongly activated ester.; and

the stereochemical configuration at the positions 2, 3, 5 and 7, and of the carbon bearing R₁ (if R₁ is not H) can be any of the 32 combinations of (R) and (S).

In an additional embodiment, the present invention provides compounds having the formula

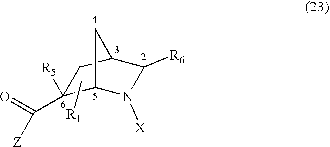

where:

X represents a first amine protecting group;

Y represents a second amine protecting group;

Z represents a weak leaving group;

R₁ represents an H, or a functional group, and can be attached to the molecule at positions 2, 3, 4, 5 or 7;

R₂ represents an H or a functional group;

R₅ represents N₃ or NR₂Y;

R₆ represents a carboxylic acid or a strongly activated ester; and

the stereochemical configuration at the positions 2, 3, 5 and 6, and of the carbon bearing R₁ (if R₁ is not H) can be any of the 32 combinations of (R) and (S).

In an additional embodiment, the present invention provides compounds having the formula

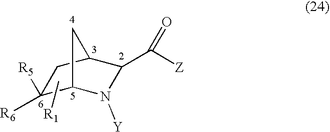

where:

X represents a first amine protecting group;

Y represents a second amine protecting group;

Z represents a weak leaving group;

R₁ represents an H, or a functional group, and can be attached to the molecule at positions 2, 3, 4, 5 or 7;

R₂ represents an H or a functional group;

R₅ represents N₃ or NR₂X;

R₆ represents a carboxylic acid or a strongly activated ester; and

the stereochemical configuration at the positions 2, 3, 5 and 6, and of the carbon bearing R₁ (if R₁ is not H) can be any of the 32 combinations of (R) and (S).

As used herein, the term “amine protecting group” refers to a moiety that protects the nitrogen of interest from attack during synthesis, and which can be easily removed at a later stage during formation of the desired compound of interest. Protecting groups are well known in the art, and include, for example, the protecting groups described in the book “Protective Groups in Organic Synthesis” by Theodora W. Greene and Peter G. M. Wuts, John Wiley and Sons publisher. Any suitable amine protecting group can be used. Preferred amine protecting groups include compounds such as 9-fluorenylmethyl carbamate, allyl-carbamate, benzyl-carbamate, substituted benzyl-carbamates, t-Butyl carbamate, 1-adamantyl carbamate, 2-nitrobenzenesulfonyl, triphenylmethyl, and (4-methoxyphenyl)diphenylmethyl and 9-phenylfluorenyl and the like. Preferably, two different amine protecting groups will be used, but under certain conditions it may be desirable to use the same amine protecting group in a given synthesis.

As used herein, the term “weak leaving group” refers to those moieties that can protect the carboxylate from attack when the monomer is first coupled to the scaffold, and leaves only upon rigidification. Such leaving groups are well known in the art. Preferably, the leaving group will mildly activate the carboxylate, so that when the monomer is in the completed chain, the nearest amine will attack the carboxylate spontaneously to form the diketopiperazaine bond. Any suitable leaving group can be used, and non-limiting examples include short chain alkoxides, thiolates, azide and sulfonamides. Thiolates include thiolates formed from short chain thiols such as ethanethiol, methanethiol, thiophenol and substituted thiophenols. Preferred weak leaving groups include fragments such as alkoxides formed from methanol, ethanol, 2-fluoroethanol, 2,2-difluoroethanol, 2,2,2-trichloroethanol, 2,2,2-trifluoroethanol, phenol, and substituted phenols.

As used herein, the term “strongly activated ester” will be used to refer to esters such as a pentafluorophenyl ester (—COOPfp), N-hydroxysuccinimide ester (—COONHS), symmetric anhydrides or asymmetric anhydrides, or other similar esters, that are rapidly attacked by amines in a bimolecular reaction under the typical conditions used in amide bond formation.

As used herein, the term “functional group” refers to any moiety that can provide additional functionality beyond that provided in the basic monomer. As an analogy, each naturally occurring amino acid contains a functional group that confers a unique set of properties, such as size, reactivity, charge, and the like, on the side-chain of each amino acid. Any functional group can be used, to confer the desired properties, so long as the use of such group does not interfere with oligomer formation. Suitable functional groups include, for example, but are not limited to, an alkyl group, a lower alkyl group, an alkoxy group, a cycloalkyl, a heterocycloalkyl, an aryl group, a heteroaryl group, an alkoxyaryl group, an aralkyl group, an aralkoxy group, an alkylthiogroup, an arylthiogroup, an alkylamido group, an alkylsulfinyl group, an alkylsulfonyl group, an alkacyl group, an alkylsulfoxide group, a halogen and a nitro group, as those terms are understood in the art.

The term “alkyl” refers to straight and branched chain alkyl groups having one to 50 carbon atoms. The term “lower alkyl” refers to an alkyl having from 1 to 8 carbon atoms.

The term “cycloalkyl” refers to saturated carbocycles having from three to twelve carbon atoms, including bicyclic and tricyclic cycloalkyl structures. A “hetero-cycloalkyl” group refers to a monocyclic radical containing carbon atoms, preferably 4 or 5 ring carbon atoms, and at least one heteroatom selected from nitrogen, oxygen and sulfur, and having no unsaturation.

The term “aryl” and “heteraryl” refer to monocyclic and polycyclic unsaturated or aromatic ring structures, with “aryl” referring to those that are carbocycles and “heteraryl” referring to those that are heterocycles. Such moieties may be optionally substituted with on or more suitable substituents, for example, a halogen, a lower alkyl (C₁-C₈), OH, NH₂, CN, COOH, O-lower alkyl, and the like.

Any of the above functional groups can optionally be substituted with any suitable substituent, including a halogen, lower alkyl group, -aryl, —OH, —NO₂, —CN, —CO₂, —O-lower alkyl, and the like. This list is non-limiting, and any substitution can be used, provided that the substitution does not interfere with the ability of the functional group to confer the desired properties on the monomer.

As can be seen in the above formulas, none of the compounds described have two carboxylic acids. It is important to have one and only one carboxylic acid (—COOH) or strongly activated ester such as a pentafluorophenyl ester (—COOPfp), an N-hydroxysuccinimide ester (—COONHS), a symmetric or an asymmetric anhydride. This is to enable the building block to be coupled to a growing macromolecule in the synthesis of bis-peptides. Bis-amino acids with two stable esters are unable to couple and useless for the synthesis of bis-peptides. Bis-amino acids with two carboxylic acids or two strongly activated esters will couple indiscriminately through both groups and create useless mixtures of products.

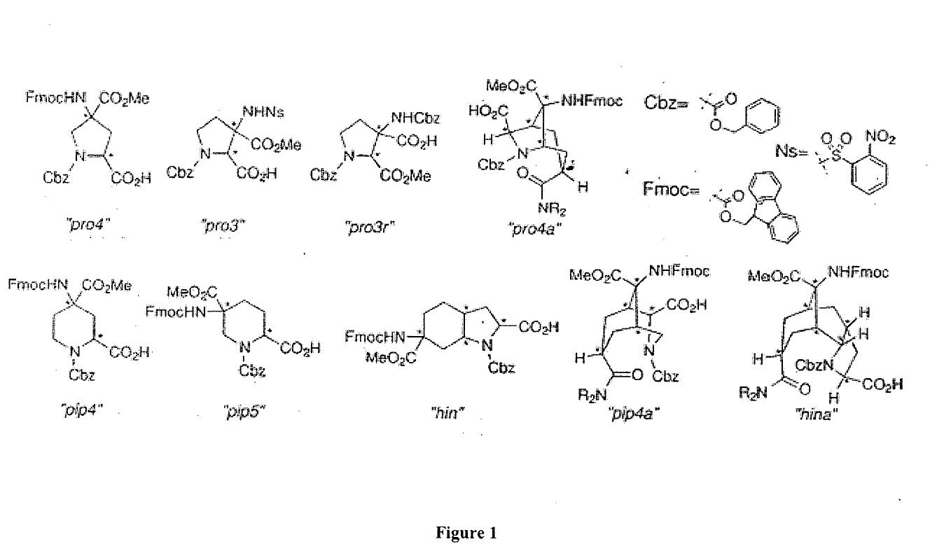

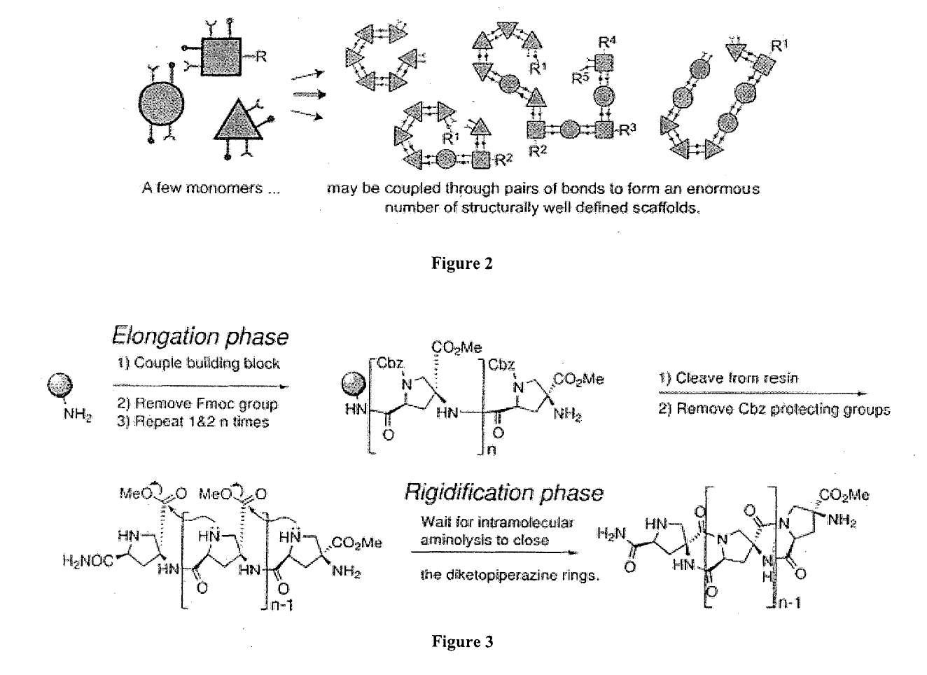

The present invention provides a collection of bis-amino acids that are the molecular building blocks of a unique methodology. Each building block has a unique rigid three-dimensional structure and contains multiple stereocenters (each indicated with “*” in FIG. 1). The building blocks are grouped in classes. The members of a class have identical constitution but vary in their stereochemistry. Each class contains at least two stereoisomers that must be synthesized or isolated in stereochemically pure form. Many of the bis-amino acids shown can be synthesized by following the same basic strategy. The strategy is to start from a stereochemically pure cyclic intermediate that contains a protected α-amino ester and a ketone. The ketone may then be converted to a hydantoin using a Bucherer-Bergs reaction and the hydantoin is then hydrolyzed and converted into another suitably protected amino ester. The Bucherer-Bergs reaction produces at most a mixture of two diastereomers that can be separated and carried individually through to form two valuable diastereomeric building blocks. A modified Corey-Link reaction[21] may also be used to convert the ketone into an azido-ester that is equivalent to a protected amino ester. The modified Corey-Link reaction also creates a mixture of two diastereomers that are separated and carried individually through to form two valuable diastereomeric building blocks. This strategy is non limiting, many other approaches can be envisioned for the synthesis of bis-amino acid building blocks.

Ideally, scaffold assembly takes place in two phases (FIG. 3). First, the building blocks are coupled on solid support through amide bonds as a flexible chain in an “elongation phase”. Then the chain is cleaved from solid support and a second set of amide bonds are formed in parallel in the “rigidification phase”. The result is a spiro-ladder oligomer with a complex and well defined three-dimensional shape that is determined by the sequence of its monomers. Solid phase peptide synthesis has made it possible to routinely synthesize peptides with lengths in excess of 40 amino acids with excellent yields[28]. Similarly, it is possible to synthesize molecules with defined shapes in the range of 1,000 to 10,000 Da. Making ten-mers with just four building blocks, 4¹⁰ or about 1,000,000 different rigid macromolecular shapes can be constructed. The synthesis of every one of these million different molecules will be quick and will follow exactly the same synthetic steps (but using different building blocks) on solid support. Alternatively, scaffolds can be assembled in solution. “Rigidification” can also be carried out at the same time the chain is being elongated.

As will be known to one skilled in the art, the process of optimizing the synthesis of the bis-amino acid monomers involves modifying reaction conditions to improve yields, modifying workup conditions to improve purity and recovery of products, eliminating chromatographic steps, developing crystallization procedures to isolate pure products and developing alternative routes to intermediates that reduce cost and improve yields of the final bis-amino acid products.

In an additional aspect, the present invention provides a method of synthesizing bis peptides comprising the steps of:

1) providing a solid support;

2) activating a first bis amino acid or naturally occurring amino acid;

3) attaching the bis amino acid or naturally occurring amino acid to the support;

4) removing the leading edge amine protecting group if a bis amino acid is used, or the amine protecting group if a naturally occurring amino acid is used;

5) activating and attaching a next bis amino acid or a next naturally occurring amino acid to the leading edge amine of the bis amino acid or amine of the naturally occurring amino acid; and

6) repeating steps 4 and 5 as necessary to achieve the desired chain length;

7) detaching the synthesized bis peptide from the support; and

8) isolating the synthesized bis peptide,

where the bis peptide synthesized in the above manner has at least two contiguous bis amino acids, and a rigidification step is carried out either after step 4 or after detachment of the bis peptide from the solid support. Optionally, the method further comprising the step of modifying or adding a functional group, after step 5. Each of these steps is more fully described below.

In the synthesis of bis-peptides, natural and unnatural amino acids can be introduced into the bis-peptide structure to confer additional properties. Only those macromolecules, that can be synthesized by this process, that contain at least two contiguous bis-amino acids and undergo rigidification to form a pair of bonds between the two contiguous bis-amino acids, are considered within the scope of the present invention.

Bis-peptide synthesis on solid support takes place by the following process:

A suitable solid phase support is prepared or purchased, such as a polystyrene resin purchased from Novabiochem for solid phase synthesis of peptides and organic compounds. Other resins can also be used. A wide variety of linkers can be used, preferred linkers are those derived from 4-hydroxymethylpheoxyacetic acid (HMPA), linkers derived from 4-hydroxymethylbenzoic acid (HMBA), RINK linkers, silylalkyl linkers, and 4-sulfamylbenzoyl based linkers.

If the resin/linker combination carries a temporary protecting group, it is removed using appropriate reaction conditions.

A protected bis-amino acid is chosen from the collection of monomers described above or a protected amino acid is chosen; this chosen monomer is activated and coupled to the solid support. Any of the bis amino acids of the present invention, or naturally occurring amino acids, can be used, and are within the scope of the present invention. If the monomer is in a pre-activated form, such as a pentafluorophenyl ester, it is first dissolved in a suitable solvent and base is added after which the mixture is added to and incubated with the resin. If the bis-amino acid is in the free-acid form, an activating agent is added with solvent and base and then the mixture is added to and incubated with the resin. Preferred solvents are N,N-dimethylformamide, 1-methyl-2-pyrrolidinone, N,N-dimethylacetimide and methylene-chloride in an appropriate ratio to ensure solubility. Preferred bases are diisopropylethylamine and triethylamine. As used herein, the term “activating agent” refers to those activating agents known to one skilled in the art and used in peptide synthesis. These are available, for example, from Novabiochem. Preferred activating agents are those that lead to the formation of 1-hydroxy-7-azabenzotriazole (HOAt) esters, N-hydroxysuccinimide (HOSu) esters, N-hydroxybenzotriazole esters (HOBt), acid fluorides. Other activation reagents such as benzotriazole-1-yl-oxy-tris(dimethylamino)-phosphoniumhexafluorophosphate (BOP), 1,1-carbonyl-diimidazole (CDI), N,N-dialkylcarbodiimides, bromo-tris-pyrrolidino-phosphonium hexafluorophosphate (PyBroP) can also be used. An excess of activated monomer is used to ensure a high yield of coupled product. Typically two to four equivalents are added. Sufficient time is allowed for the coupling to take place, typically 10 to 120 minutes.

The resin is then subjected to several wash cycles to remove byproducts and to prepare the resin for the next operation. The preferred method of washing is three five minute cycles of a solvent swelling solvent such as N,N-dimethylformamide followed by three five minute cycles of a solvent shrinking solvent such as isopropanol or methanol, followed by three five minute cycles of the solvent swelling solvent.

At this stage, the decision may be made to modify the monomers on the resin to attach additional functionality or modify functionality. Preferred modifications are alkylation of sulfonamide protected amines or oxidation, reduction, alkylation, acylation or amidation of the appended R₁ functional group described in the monomer claims. Adding a functional group can be carried out after the adddition of each new monomer, as desired.

The leading edge amine protecting group is removed from the most recently attached monomer. The resin is subjected to reaction conditions appropriate to remove the temporary protecting group without cleaving the growing chain from the resin. If the leading edge amine is masked as an azide, the azide is reduced to liberate the amine.

If the monomer is a bis-amino acid then the temporary protecting group on the leading edge amine is removed using conditions appropriate for the temporary protecting group. The leading edge amine is defined as follows. Each monomer carries two protected alpha-amino acids. The leading edge amine is the amine on the monomer that is not directly connected to the carbon alpha to the activated ester is now attached to the resin. In other words, the leading edge amine is the amine alpha to the carbonyl carrying the weak leaving group.

If the monomer is a regular protected amino acid then the temporary protecting group is removed from the amine.

The resin is then subjected to several wash cycles to remove byproducts and to prepare the resin for the next operation. The preferred method of washing is three five minute cycles of a solvent swelling solvent such as N,N-dimethylformamide followed by three five minute cycles of a solvent shrinking solvent such as isopropanol or methanol, followed by three five minute cycles of the solvent swelling solvent.

The next monomer is activated and attached, as described above, with as many repetitions of these steps as necessary to achieve the desired chain length.

At this stage the decision may be made to rigidify one or more of the contiguous bis-amino acids in the growing chain while it is still attached to the resin. This would be carried out by removing the protecting group from the trailing edge amine (the amine connected to the alpha carbon shared by the carbonyl that was first attached to the resin). After removal of the protecting group, the resin is incubated under either neutral, basic or acidic conditions to promote the multiple, parallel, intramolecular aminolysis reactions that rigidify the scaffold. As used herein, the terms “rigidify” or “rigidification” will be used to refer to this two-step process.

The activation and attachment steps are repeated for every monomer that is to be incorporated into the growing macromolecule. As the final monomer is coupled, the decision may be made to leave the last leading edge amine protecting group on the resin during cleavage.

Once the last monomer has been added, the decision may be made to attach a final group to the resin through the last leading edge amine. Examples of useful modifications would be to treat the resin with dansyl-chloride to attach a fluorescent dansyl group to the resin, or to treat the resin with activated carboxylic acids.

At this stage the decision may be made to rigidify one or more of the contiguous bis-amino acids in the growing chain while it is still attached to the resin. This would be carried out by removing the protecting group from the trailing edge amine (the amine connected to the alpha carbon shared by the carbonyl that was first attached to the resin). After removal of the protecting group, the resin is incubated under either neutral, basic or acidic conditions to promote the intramolecular aminolysis reactions that rigidify the scaffold.

The new macromolecule is then cleaved from the solid support using cleavage conditions appropriate for the linker/resin on which the scaffold was assembled. The macromolecule will then be isolated and if necessary, purified using reverse phase high pressure liquid chromatography. A preferred method of isolation of the macromolecule from cleavage byproducts is to rapidly dilute the cleavage mixture into diethyl ether and isolate the product by centrifugation. A preferred situation is to use an acid cleavable linker and acid cleavable protecting groups on the trailing edge amines. Strong acid cleavage conditions such as hydrogen fluoride or trifluoromethanesulfonic acid will simultaneously cleave the macromolecule from the resin and strip all of the acid cleavable protecting groups from the macromolecule preparing it for rigidification by incubation under conditions that promote intramolecular aminolysis.

Bis-amino acids that require rigidification at this stage and where the trailing edge amine protecting groups have not been removed under the resin cleavage conditions, will be rigidified by removing the protecting group from the trailing edge amine (the amine connected to the alpha carbon shared by the carbonyl that was first attached to the resin). After removal of the protecting group, the resin is incubated under either neutral, basic or acidic conditions to promote the intramolecular aminolysis reactions that rigidify the scaffold. For example, if Cbz groups are used as the trailing edge amine temporary protecting group and they have not been removed by strong acid cleavage of the linker, they can be removed at this point using hydrogenolysis with hydrogen gas on palladium catalyst. A preferred method of promoting diketopiperazine formation through intramolecular aminolysis is to incubate the macromolecule in 20% piperidine in dimethylformamide for 48 hours. Basic conditions are known to promote epimerization of diketopiperazines so care must be taken. Slower, acidic conditions can also be used and they are known to avoid epimerization of diketopiperazines.

The rigidified scaffold is isolated and may be purified by reverse phase high pressure liquid chromatography. The preferred method of isolating the rigidified scaffold is by rapidly diluting a solution of the macromolecule into diethyl ether followed by centrifugation of the resulting precipitate.

A solution phase synthesis is also within the scope of the present invention. This method provides a method of synthesizing bis peptides comprising the steps of:

1) providing a bis-aa or bis-peptide fragment containing a mixture of bis-aa and naturally occurring aa. with an unprotected leading edge amine and a protected trailing edge carboxylic acid;

2) providing a bis-aa or bis-peptide fragment containing a mixture of bis-aa and naturally occurring aa with a protected leading edge amine and an activated ester;

3) coupling the two fragments in solution;

4) isolating the synthesized bis-peptide;

5) removing the leading edge amine protecting group or the trailing end carboxylic acid protecting group; and

6) repeating steps 1,2,3,4 to achieve the desired chain length;

where the bis peptide synthesized in the above manner has at least two contiguous bis amino acids, and a rigidification step is carried out either after step 3 or after detachment of the bis peptide from the solid support. Optionally, the method further comprises the step of modifying or adding a functional group, after step 3. Macromolecules comprised of 2, 3, 5, 10, 15, 20, 25, 50, 100, 200, 300, or 500 units synthesized by the either of the above methods (solid support or solution phase) are contemplated, with any mixture of monomer units, including mixtures of bis amino acids and naturally occurring amino acids.

Isolation of the bis peptide would be the same as described above. As used herein, the term “coupling” means to mix an unprotected amine together with an activated ester in solution and waiting for minutes to hours while the amide bond is formed. The amine attacks the activated ester in a bimolecular reaction and forces out the strong leaving group. The result is the formation of a strong, stable amide bond.

The monomer classes of formulas (15) and (16) would be synthesized starting from a suitably protected 4-oxo-pipicolic acid intermediate described in the synthesis of the pip4 monomer class and annulated in the same way as in the pro4a synthesis followed by conversion of the ketone to an amino ester using a Bucherer-Bergs reaction or a modified Corey-Link reaction.

The monomer classes of formulas (17) and (18) would be synthesized starting from a suitably protected 5-oxo-pipicolic acid intermediate described in the synthesis of the pip5 monomer class and annulated in the same way as in the pro4a synthesis followed by conversion of the ketone to an amino ester using a Bucherer-Bergs reaction or a modified Corey-Link reaction.

The monomer classes of formulas (19) and (20) would be synthesized starting from suitably protected allyl-glycine and ethynylation on the nitrogen. After a Pauson-Khand reaction and reduction of the resulting olefin, the ketone would converted to an amino ester using a Bucherer-Bergs reaction or a modified Corey-Link reaction.

The monomer classes of formulas (21), (22), (23) and (24) would be synthesized using a diastereoselective imino-Diels-Alder reaction with cyclopentadiene. The resulting disubstituted olefin would be hydroborated, and the resulting alcohol would be oxidized to a ketone. The ketone would converted to an amino ester using a Bucherer-Bergs reaction or a modified Corey-Link reaction.

The base-promoted epimerization of diketopiperazines has been studied extensively[43]. It was thought that incubation of scaffolds in base to promote intramolecular aminolysis might cause epimerization and loss of the carefully crafted stereochemical structure. It has now been found that in the reverse phase C₁₈ chromatograms of fully rigidified scaffold, traces of compounds with identical molecular weights to the scaffolds appear, but with slightly different retention times. It is thought that these are diastereomeric scaffolds. However they collectively represent less than five percent of the overall material, and could originate from stereochemical impurities in the bis-amino acid building blocks. The absence of significant amounts of diastereomeric impurities demonstrates that diketopiperazine epimerization under the 20% piperidine/DMF conditions is much slower than intramolecular aminolysis.

Synthesis of Functionalized Building Blocks

Given the bis-amino acids that are disclosed above, it is straightforward to uniquely functionalize the two ends of any poly-bis-peptide. To display more than two functional groups, monomers will be developed that not only contribute to the shape of a scaffold, but also display a selected functional group in the same way that a natural amino acid displays its functional side chain. A general approach to this would be to a-alkylate any of the N-protected amino ester ketones just prior to the Bucherer-Bergs reaction. In the prototypical pro4a monomer class, the intermediate ketone will be α,αdialkylated to create a monomer that forms turns in poly-bis-peptides and displays an additional functional group (FIG. 9).

### EXAMPLES

The following examples are intended to illustrate the invention and should not be construed as limiting the invention in any way.

### Synthesis of the Pro4 Monome Class

### Example 1

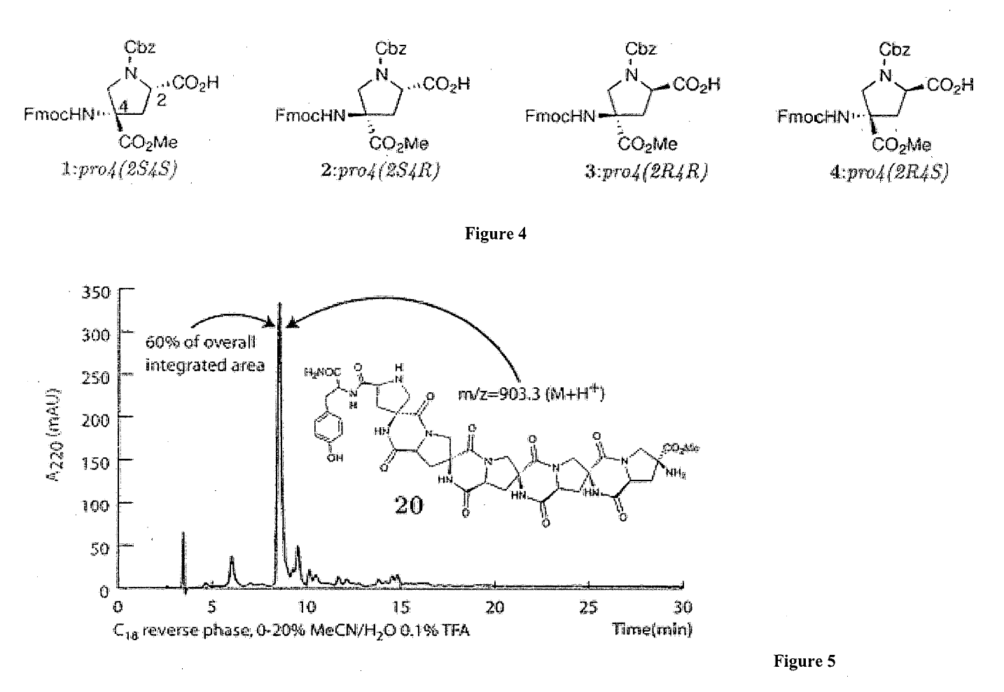

These monomers were chosen as prototypes because they are capable of forming macromolecules shaped like rods, circles, figure-eights. etc. (FIG. 4) and because they can all be made from inexpensive, commercially available trans-4-hydroxy-L-proline 5. The first member of this class 1 was synthesized on a 1.8 gram scale in nine steps with an overall yield of 20% (Scheme 1).

The synthesis began by protecting the amine of 4-hydroxyproline 5 as a benzyl carbamate (N-Cbz) followed by oxidation of the secondary hydroxyl to form the ketone 6. The carboxyl group was then protected as a tert-butyl ester to form 7. A Bucherer-Bergs reaction[31] was carried out to install a quaternary stereocenter and form the diastereomeric hydantoins 8 and 9 with a diastereoselectivity[32] of 5:1. Using a single chromatographic column, 16.5 grams of pure diastereomeric hydantoin 8 was isolated from a 20 gram mixture of 8 and 9. The hydantoin 8 was then hydrolyzed using a mild, two-step procedure developed by Rebek and co-workers [22] and the amino acid 10 was isolated in excellent yield. The free amine was then protected as the 9-fluorenylmethyl carbamate (Fmoc) and the carboxylate converted to the methyl ester to form the fully protected building block 11. Finally, the tert-butyl ester was deprotected with trifluoroacetic acid and the building block with its free carboxylic acid is used without further purification in solid phase couplings.

The minor hydantoin 9 has been carried to within one step of the building block 2:pro4(2S4R). The reported yields are unoptimized (Scheme 2).

### Example 2

Synthetic access to the two additional stereoisomers of the pro4 monomer class has been established. Using a controlled epimerization procedure[34], 30 grams of trans-4-hydroxy-(L)-proline 5 were converted to the diastereomer cis-4-hydroxy-(D)-proline 14 with 57% isolated yield. By carrying this material through the synthesis described in Scheme 1, synthesis of the other two enantiomers of the pro4 building block class 3:pro4(2R4R) and 4:pro4(2R4S) can be accomplished. (Scheme 3)

### Synthesis of Scaffolds

### Example 3

Pure three-mer scaffolds and five-mer scaffolds with extended rod-like structures in useful amounts have been synthesized. Three units of building block 1:pro4(2S4S) (Sequence 1) were assembled to form the molecular rod 19 using sequential solid phase synthesis on a 46 μmole scale (Scheme 4). The synthesis took place on an AM resin with a Rink Amide linker available from Novabiochem. Each building block was activated as the 1:pro4(2S4S)-hydroxy-7-azabenzotriazole (HOAt) ester [23] and quantitative coupling to the previous building block was achieved in less than 10 mm; a surprising result given the apparent hindered nature of the nucleophile. After coupling three monomers, an Fmoc-Tyr(t-Bu)-OAt residue was coupled to increase the hydrophobicity of the final scaffold so that it would bind to a C₁₈ column. After removal of the tyrosine Fmoc group, the amine terminus rapidly attacked the adjacent methyl ester to form a diketopiperazine 18 (indicated in the sequence as cyclo-(Tyr)). The flexible oligomer 17 was cleaved from the resin and its mass was confirmed by reverse phase liquid chromatography with mass spectrometry (RP-LCMS). The carboxybenzyl (Cbz) groups were then removed by hydrogenolysis to obtain 18. The flexible oligomer 18 was converted into the rigidified scaffold 19 through the parallel formation of two diketopiperazine [24] rings by exposure to 20% piperidine/DMF over 48 hours at 4° C.[25] The product precipitated from the 20% piperidine solution and filtration provided 5 mg of 19 (˜15% yield based on resin loading). The scaffold 19 was soluble in water to more than 5 mg/ml and stable in neutral and acidic aqueous solution at room temperature for more than three weeks. A previous three-mer synthesis using alanine in the place of tyrosine resulted in a scaffold that was so polar that it refused to bind to a C₁₈ column in pure 0.1% TFA/H₂O and eluted in the flow-through. Solubility was measured by dissolving the compound in 0.1% TFA, centrifuging it, injecting the solution into an HPLC and integrating the resulting peak relative to a tyrosine standard. For more accurate solubility measurements sedimentation equilibrium can be used to determine the poly-bis-peptide molecular weight in solution [26].

The predicted structure of 19 is consistent with the solution structure. To construct a model of the scaffold 19, an in vacuo conformational search was carried out using the AMBER95 [27] force field within the molecular mechanics package MOE.[28] The conformational search revealed a cluster of five lowest energy conformations all within 0.4 kcal/mol of each other separated from the next highest energy cluster by a gap of 2.2 kcal/mol. Calculating the populations of the 24 lowest energy conformations using the Boltzmann equation suggests that the molecule will spend more than 95% of its time collectively in the five lowest energy minima. A superposition of these predicted lowest energy conformations reveals that rings B, C, D, E, F and the folded tyrosine conformation are identical. The differences between the five conformations involve combinations of rotamers around the C2-C3 bond, rotamers around the tyrosine OH bond and two envelope conformations of ring A. The 2D ROESY spectra display cross-peaks consistent with the predicted rigid B through F ring system. The NMR data are more consistent with ring A existing predominately in the single envelope conformation based upon a strong cross-peak between 4H and 10H and a weak or non-existent cross-peak between 4H and 10H.

### Example 4

To demonstrate the generality of this synthetic approach, the five-mer scaffold 20 (FIG. 5) was synthesized in a similar fashion to scaffold 19. The resin (49 mg, 31.4 umol loading) was first charged with an Fmoc protected tyrosine residue and then five cycles of coupling with monomer 1:pro4 (2S4S) were performed. Roughly 13 mg of product resin was removed and subjected to the TEA cleavage conditions. The Cbz groups were removed and the scaffold was rigidified by exposure to 20% piperidine/DMF over 24 h. In this case, 3 mg of the scaffold 20 (˜33% from initial resin loading) was isolated by precipitation with ether and centrifugation. After all of these manipulations, this unpurified material was highly homogeneous (FIG. 5), and HPLC-MS analysis confirmed that the major peak has the expected mass. This material was soluble in 10% D₂O/H₂O at 5 mg/ml. The in vacuo minimum energy structure suggests that the spiro-fused ring structure forms a narrow left handed helical rod with approximately four residues per turn and a pitch of ˜20 Å.

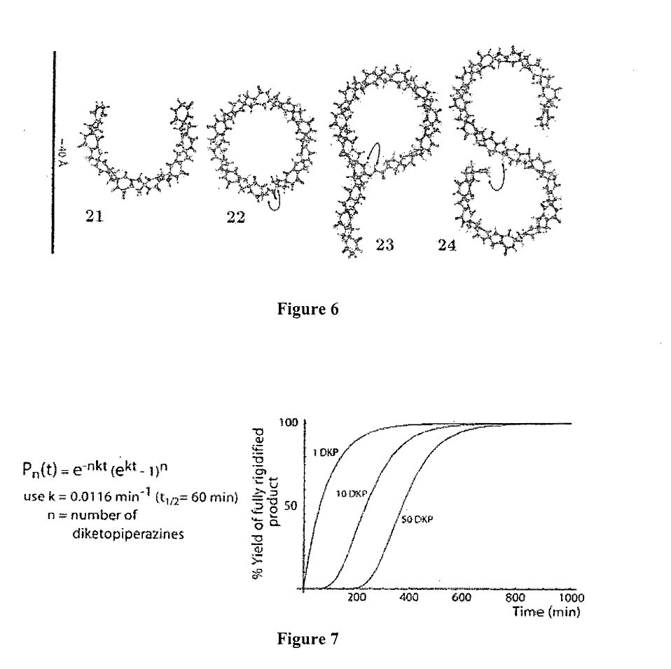

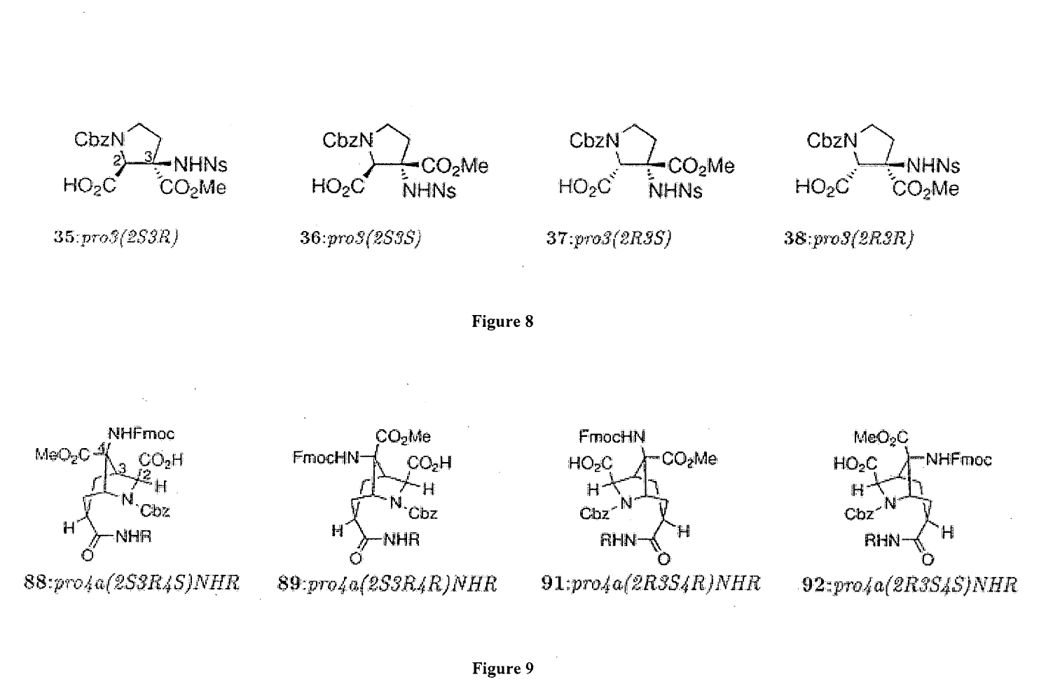

The sequences of four of these scaffolds are shown (Sequence 2, FIG. 6). These particular sequences form interesting shapes that in three cases create close contacts between monomers that are widely separated in sequence. Hundreds of crystal trials using a few milligrams of material[40] can be set up with protein crystallization methods. With 5 mg of a scaffold, a 500 μl solution at 10 mg/ml concentration can be prepared and used to set up 250 2 μl sitting drops for crystallization using the vapor diffusion technique. The solution structures of the compounds 21, 22 and 24 where predicted close contacts are expected to give rise to specific ROESY cross-peaks can also be determined. Each synthesis will be performed on an Applied Biosystems 433A peptide synthesizer. This instrument is capable of performing syntheses on a 20 μmol scale, requiring only 5 equiv. of building block per coupling (˜60 mg of bis-amino acid/coupling) providing between 7 mg and 16 mg of the bis-peptides 21 and 24 respectively (conservatively assuming 33% recovered yield from resin).

In the event that NMR structure determination is hampered by overlapping signals, ¹⁵N and ¹³C can be site-selectively incorporated to simplify NMR structure determination. Incorporation of ¹⁵N and ¹³C isotopes into proteins is a common technique used to enhance spectral resolution in the determination of protein structures by NMR [29]. These isotopic labels can be used in the Bucherer-Bergs reaction through the use of inexpensive ¹⁵N labeled ammonia and ¹³C labeled potassium cyanide. To further facilitate NMR structure determination, ¹⁵N labeled and unlabeled bis-amino acids can be mixed at the point where coupled to solid support. This allows rapid assignment of resonances to positions within the sequence by the relative NMR signal intensity within the ¹⁵N spectra.

Improvements in solid-phase assembly techniques now permit routine synthesis of long (>40 residues) complex peptides [30]. However, some peptide syntheses experience difficult couplings when they are elongated through residues 12-20 of their sequences [31]. These difficult couplings are believed to be due to β-sheet formation on solid support. The monomers of the present invention should not be capable of forming β-sheets. Difficult couplings can be addressed by using additional couplings, changing solvents, increasing the temperature and by using more active coupling reagents [31]. Switching to different resins and lower loading resins can also help to avoid coupling problems [31].

Increasing the length of the scaffolds is expected to have a minimal impact on the “rigidification phase” of scaffold synthesis (FIG. 3). The kinetics of diketopiperazine closure in scaffolds of arbitrary length are readily modeled. Assuming that every ring closes independently and with the same rate constant, the function that describes the formation of fully closed product is Pₙ(t)=e^{-nkt}(e^{kt}−1)ⁿ (FIG. 7). In this equation “n” is one less than the number of building blocks and “k” is the rate constant of diketopiperazine closure. The equation predicts that 532 min is required to achieve 99.9% yield of a fully rigidified 2-mer scaffold-and only 932 mm to achieve the same yield of a fully rigidified 51-mer scaffold. If longer scaffolds do not completely rigidify, as indicated by LCMS analysis of the final product, tandem-mass-spectrometry can be used to fragment the scaffold at any single amide bond. This will allow pinpointing slow closing diketopiperazines and acceleration of their closure by replacing their methyl esters with more active esters such as β-fluoro-ethyl esters or electron withdrawing group substituted benzyl esters.

### Synthesis of the Pro3 Monomer Class

### Example 5

Trans-3-Hydroxy-(L)-proline 39 is commercially available from Acros at $19/gram (2000/2001 catalog) and is the starting point for two diastereomeric members of the pro3 monomer class. First the carboxylate of 39 will be converted to the methyl ester 40 via a Fischer esterification. Next, the amine will be protected using the 9-phenyl-fluorenyl (PhF) group [32] to obtain 41. The alcohol will then be oxidized to the ketone 42 using a TPAP oxidation. This sequence is similar to that used by Kamenecka and co-workers in their enantioselective synthesis of 3-substituted prolines [33]. The N-PhF group will protect the a-carbon from epimerization. A Bucherer-Bergs reaction is then carried out to form the two diastereomeric hydantoins 43 and 44. In the event that only one diastereomer is formed selectively, the two step Strecker/Bucherer-Bergs sequence suggested by Edward [34] can be used to obtain the second diastereomer. In either case, access to both diastereomers 43 and 44 is obtained. The diastereomeric hydantoins can be separated by chromatography as in the pro4 monomer class or separated by selective crystallization. The two diastereomeric hydantoins will be carried through independently to form two diastereomeric building blocks. If the hydantoins cannot be separated at this stage, the synthesis can continue with the mixture, with separation of the diastereomers at a later stage.

The purified hydantoin 44 will be hydrolyzed using the method developed by Rebek and co-workers [22] to produce the free amino acid 45. The amine group is then protected as a 2-nitrobenzenesulfonamide [35] (abbreviated “Ns”) and the carboxylate converted to the methyl ester 46. The 2-nitrobenzenesulfonamide group has been developed as a temporary amine protecting group for peptide synthesis [36]. This is a convenient protecting group that can be cleanly removed in minutes with base and β-mercaptoethanol. Finally, the PhF group is replaced with a Cbz group to form 47 and selectively hydrolyze the methyl ester to the carboxylate to form the completed building block 35. Hydrolysis of the secondary methyl ester on C2 (Scheme 7) is expected to be selective over the tertiary methyl ester on C3 [35]. Epimerization of the building block during the final base hydrolysis should not be a problem because of the steric strain that would build up as C2 becomes sp² hybridized and interacts with the Cbz group.

By carrying the other hydantoin 44 through the same steps described above (Scheme 7), synthesis of the diastereomeric building block 36:pro3(2S3S) is possible. An alternative method of synthesis of the pro3 class is found in Reaction Scheme 14.

### Example 6

The starting material for 37:pro3(2R3S) and 38:pro3(2R3R), the other two stereoisomers in the pro3 class can be synthesized using an established chemoenzymatic route. Non-fermenting bakers yeast reduction of the known ketoester 48 [37] has been used to synthesize the protected cis-3-Hydroxy-(D)-proline 39 with greater than 99% ee (Scheme 8). Gellman and co-workers have used a similar bakers yeast reduction to synthesize the starting material for the synthesis of their Fmoc-AP(Boc) beta-amino acid [38]. The building blocks 37:pro3(2R3S) and 38:pro3(2R3R) can be synthesized using the same approach as was used for 35:pro3(2S3R) and 36:pro3 (2S3S) but starting from cis-3-Hydroxy-(D)-proline.

### Synthesis of the pip4 and pip5 Monomer Classes

### Example 7

Syntheses for the pip4 and pip5 monomer classes borrows much of the chemistry used in the synthesis of the pro4 class (Scheme 9). The ring expansion on the intermediate 7 using boron trifluoride etherate and ethyl diazoacetate was carried out to form the two keto esters 51 and 52 [39]. After decarboethoxylation with sodium chloride in wet dimethyl sulfoxide the two ketones 53 and 54 were separated by chromatography and subjected individually to a Bucherer-Bergs reaction. The Bucherer-Bergs reaction on 53 led to two diastereomeric hydantoins 55 and 56 in a ratio of 3:1 that were separated by chromatography.

The stereochemistry of each diastereomer can be determined using NMR and each can be carried through the same final steps as in the synthesis of 1:pro4 (2S4S) to produce two members of the pip4 monomer class. The Bucherer-Bergs reaction on 54 produced another two digatereomeric hydantoins 57 and 58 with a ratio of 3:2. After separation for these two diastereomers and assignment of their stereochemistry individually carried through to form two members of the pip5 monomer class. The enantiomers of these two monomers will be accessible through the enantiomeric ketone 15.

### Synthesis of the hin Monomer Classes

### Example 8

Synthesis for the hin monomer class begins with stereochemicaily pure N-Cbz protected L-tyrosine 59 and uses the oxidative cyclization chemistry developed by Peter Wipf and co-workers[59] to form the bicyclic N-Cbz protected methyl-ester ketone 60. A Bucherer-Bergs reaction has been carried out on 60 and obtained two hydantoins 61 and 62 with a diastereomeric ratio of 3:1. These will be separated, their stereochemistry determined, and carried through to form two members of the hin monomer class. Several other stereoisomers of this class are accessible starting from D-tyrosine and through the extremely versatile chemistry developed by the Wipf group to alter the stereochemistry of the hydroindole core[59].

### Example 9

The synthetic approach (Scheme 11, Scheme 12, Scheme 13) builds on the synthesis of the pro4 monomer class (Scheme 2). The synthesis starts with the formation of the enamine 69 from the intermediate 7. The enamine will then be carried through an α,α′ annulation procedure with allyl β,β′-dibromo-isobutyrate 70. An acetic acid workup of the annulation reaction should afford 71. As precedent, an almost identical annulation reaction has been described involving 69 and 2-chloromethyl-3-chloropropiophenone [40] with greater than 80% yield and complete diastereoselectivity. A similar annulation reaction using ethyl β,β′-dibromo-isobutyrate and the pyrrolidine enamine of cyclopentanone afforded an 80% yield of endo-3-carbethoxybicyclo[3.2.1]octan-8-one [41]. The ketone 71 can be carried forward through a Bucherer-Bergs reaction which is predicted to provide a mixture of diastereomers [34]. If the diastereoselectivity is high, the Strecker/Bucherer-Bergs two step procedure used by Edward [34] can be used to obtain the second diastereomer.

Diastereomeric hydantoins 72 and 73 can be separated using chromatography or crystallization and carried through the final steps to obtain both monomers 88:pro4a(2S3R4S)NHR and 89:pro4a(2S3R4R)NHR. To produce the functionalized monomer 88:pro4a(2S3R4S)NHR from the hydantoin 72, the hydantoin will first be acylatee with Boc-anhydride and DMAP to form 74. Then the allyl ester 74 will be deprotected with palladium and phenyl-silane to obtain 75 and introduce the desired functionality by activating the resulting carboxylate as an asymmetric anhydride and coupling it with a suitably functionalized primary or secondary amine. Any amine component can be introduced that does not interfere with the subsequent steps of building block and scaffold synthesis.

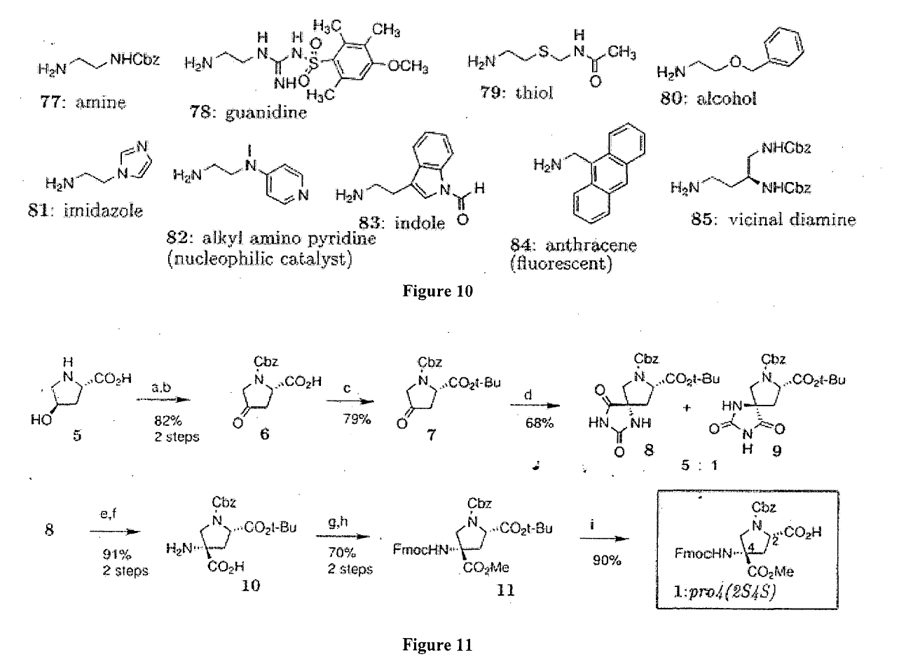

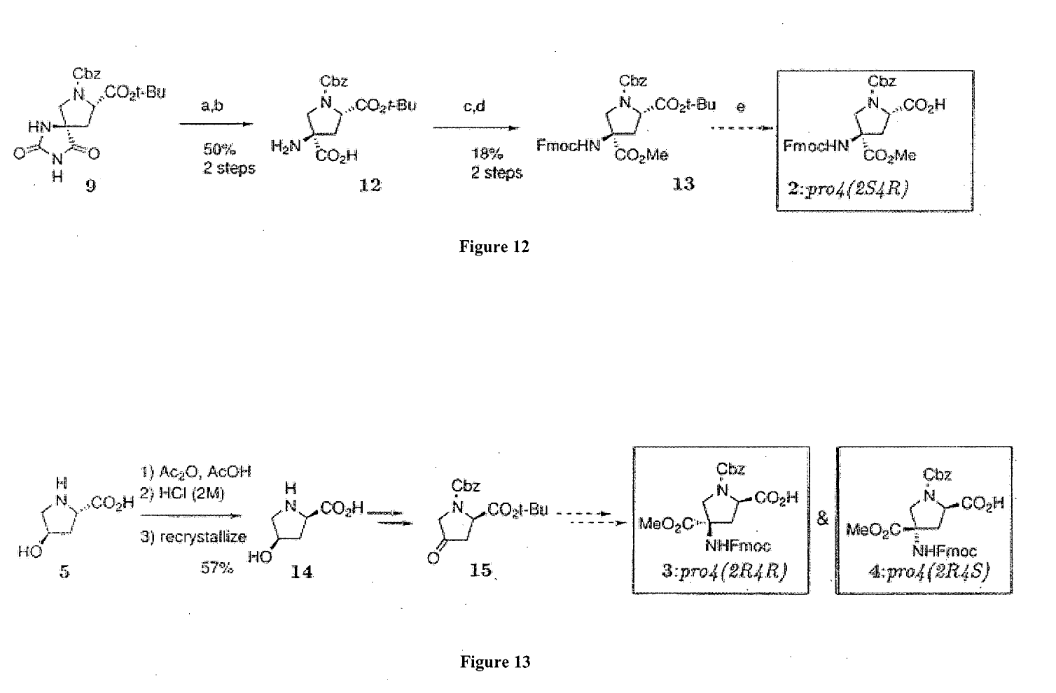

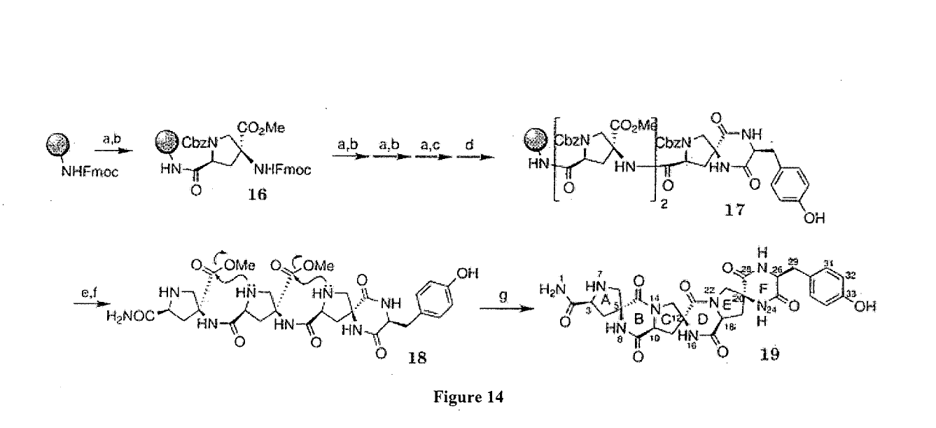

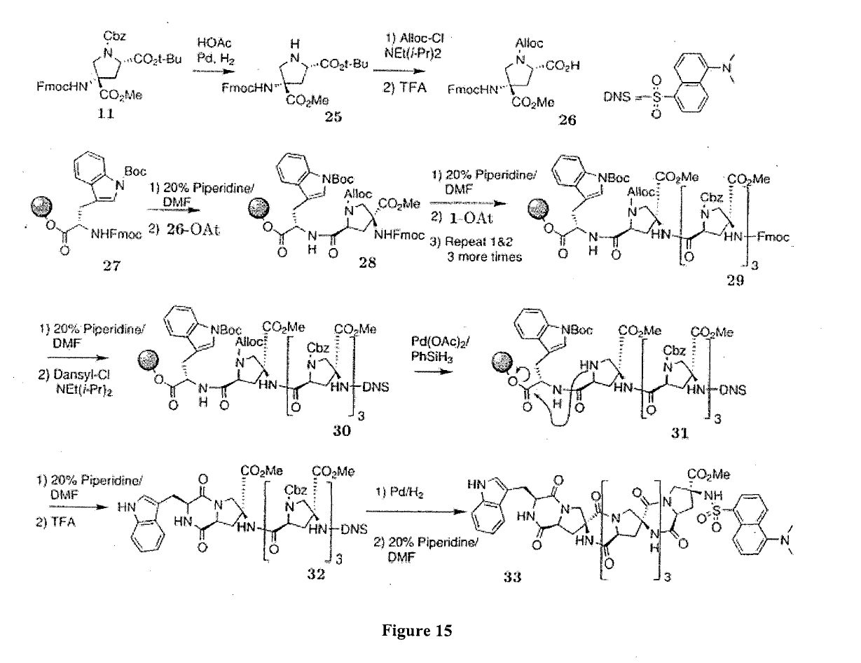

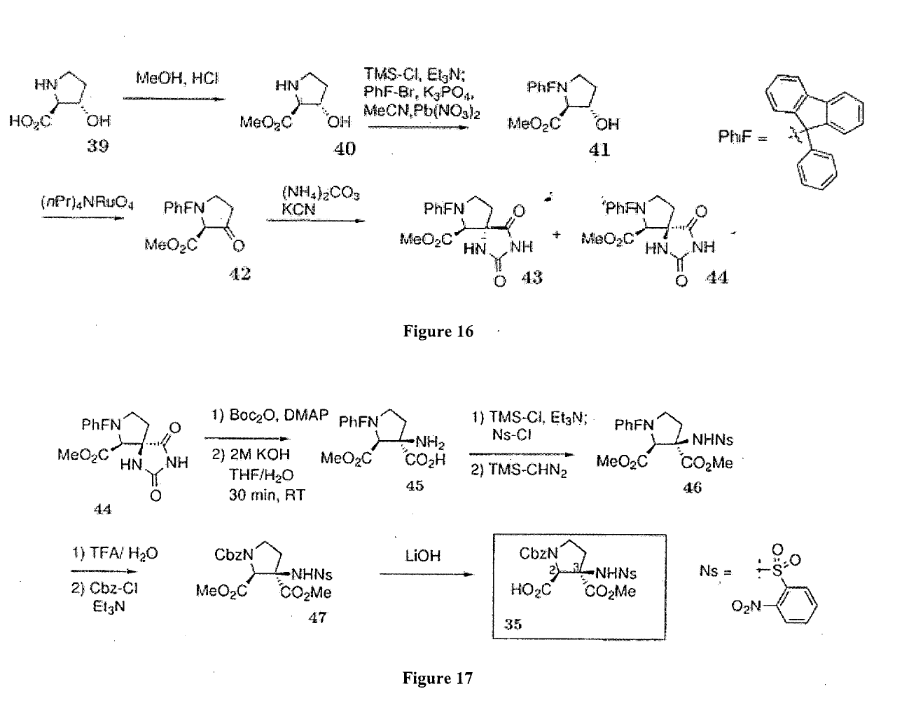

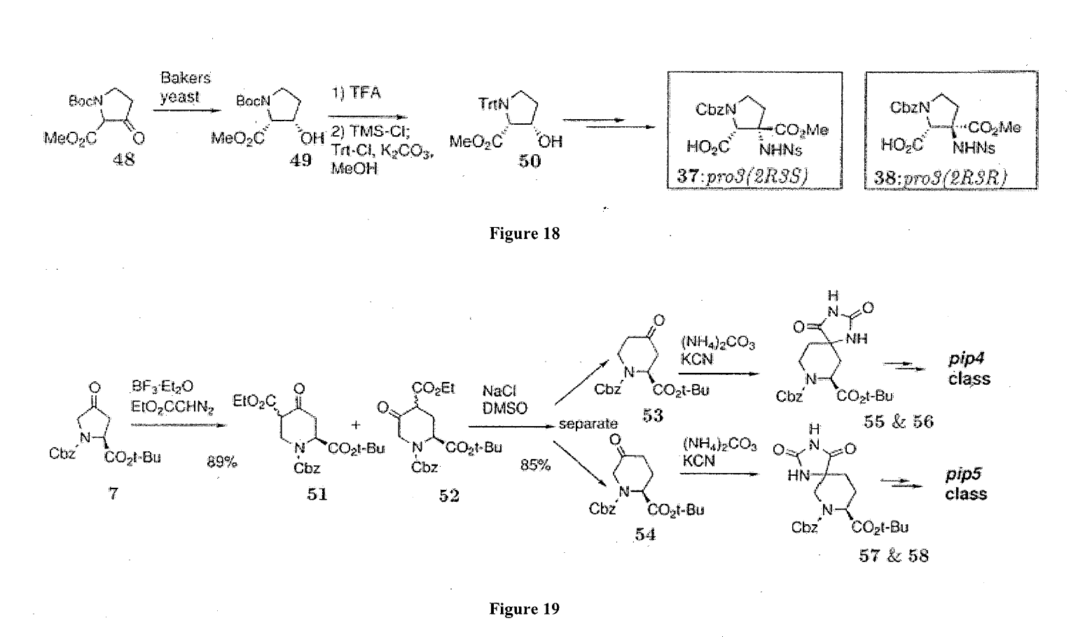

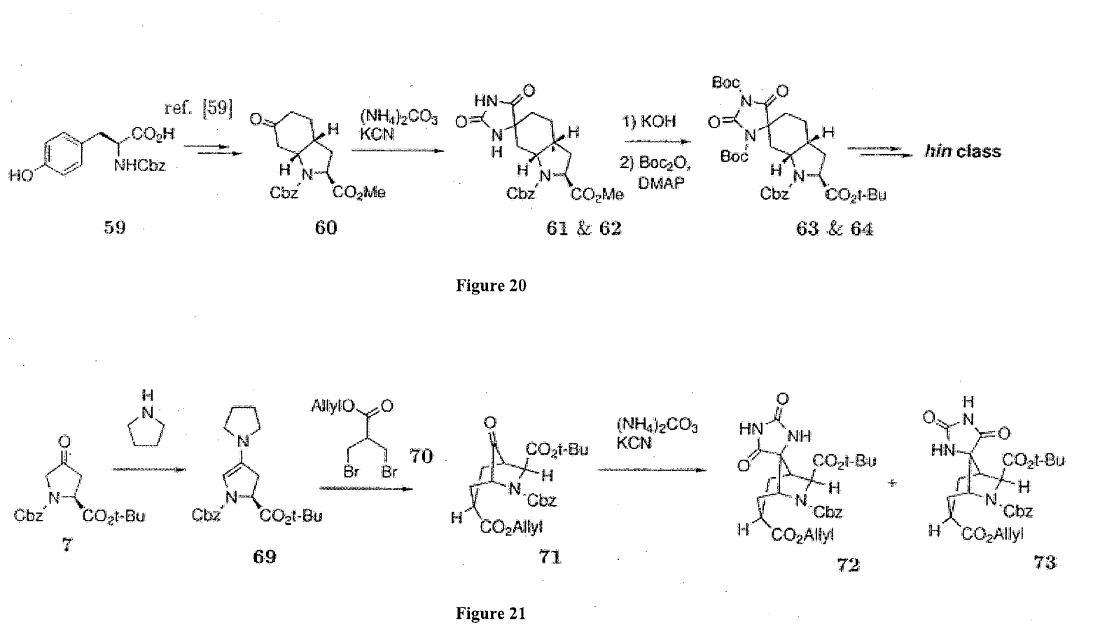

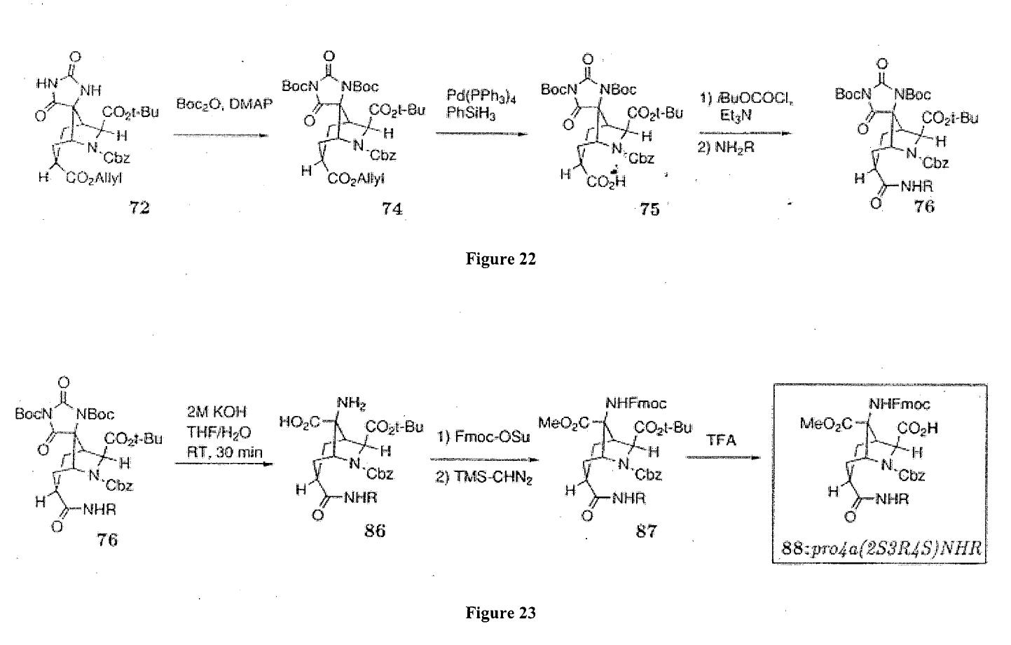

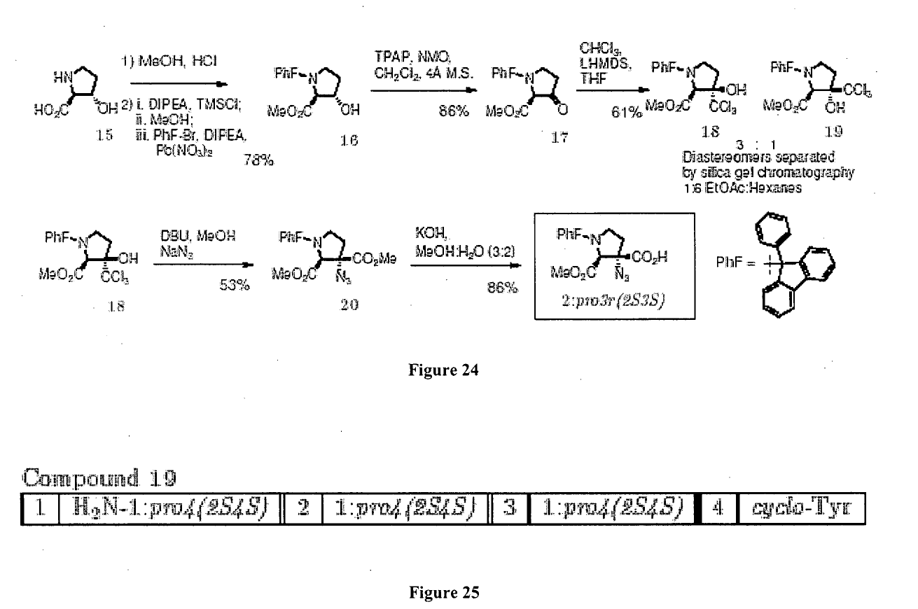

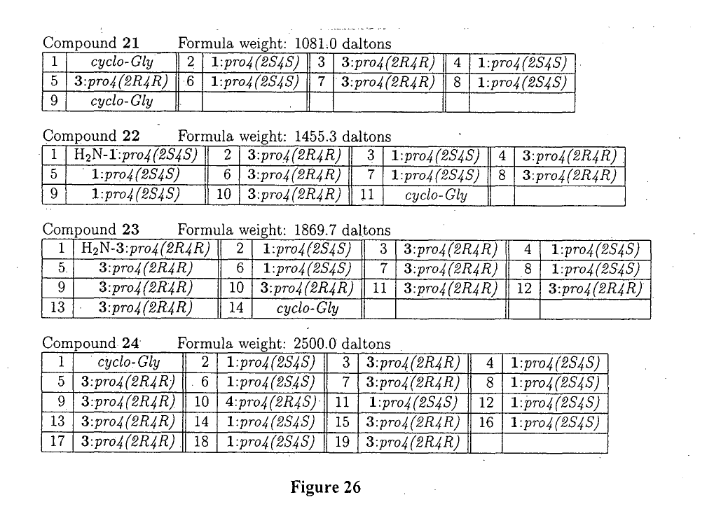

Many amines can be attached to this building block through the amide linkage to form functionalized versions of 76. Examples are shown in FIG. 10. For reactive functionality protecting groups that have been developed for side chain protection in Boc solid phase peptide synthesis were chosen. These can be removed either by hydrogenolysis or by treatment with a strong acid such as trifluoromethanesulfonic acid (TFMSA). The functional groups include, but are not limited to, those displayed by natural amino acids such as an amine 77, a guanidine 78, a thiol 79, an alcohol 80, an imidazole 81 and an indole 83. Also suitable are non-natural side chains, for example, like the dialkylamino pyridine group 82 that could serve as a nucleophilic catalyst, an anthracene group 84 that could be used to construct fluorescent sensors, and a vicinal diamine 85 that could be used to form a catalytic salen metal complex. As described above, a functional group can be any group which provides additional functionality above that provided by the basic monomer unit.

To complete this functionalized monomer 76 will be carried through the hydrolysis of the bis-Boc-hydantoin 76, N-Fmoc protection and methyl ester formation of the amino acid 86 and finally removal of the tert-butyl group of 87 to produce the finished building block 88:pro4a(2S3R4S)NHR.

Through access to 15, the enantiomer of the ketone 7, the other two enantiomers of the pro4a, 91:pro4a(2R3S4R)NHR and 92:pro4a(2R3S4S)NHR can be synthesized. Many of the other monomer classes, including the pip4 class, the pip5 and the hin class (FIG. 1) have as intermediates N-Cbz tert-butyl ester ketones that will be subject to the annulation reaction shown in Scheme 11. These will lead to many other functionalized bis-amino acids.

Whereas particular embodiments of this invention have been described above for purposes of illustration, it will be evident to those skilled in the art that numerous variations of the details of the present invention may be made without departing from the invention as defined in the appending claims.

### REFERENCES

1. DeGrado, W. F., et al., De novo design and structural characterization of proteins and metalloproteins. Ann. Rev. Biochem., 1999. 68: p. 779-819.

2. Dahiyat, B. I., C. A. Sarisky, and S. L. Mayo, De novo protein design: towards fully automated sequence selection. J Mol Biol, 1997. 273(4): p. 789-96.

3. Schafmeister, C. E., et al., A designed four helix bundle protein with native-like structure. Nature Structural Biology, 1997. 4(12): p. 1039-1046.

4. Nicolaou, K. C., et al., Total Synthesis of Brevetoxin-B .1. First Generation Strategies and New Approaches to Oxepane Systems. Journal of the American Chemical Society, 1995. 117(41): p. 10227-10238.

5. Nicolaou, K. C., et al., Total Synthesis of Brevetoxin-B .3. Final Strategy and Completion. Journal of the American Chemical Society, 1995. 117(41): p. 10252-10263.

6. Nicolaou, K. C., et al., Total Synthesis of Brevetoxin-B .2. 2nd Generation Strategies and Construction of the Dioxepane Region [Defg]. Journal of the American Chemical Society, 1995. 117(41): p. 10239-10251.

7. Kaszynski, P., A. C. Friedli, and J. Michl, Toward a Molecular-Size Tinkertoy Construction Set—Preparation of Terminally Functionalized [N]Staffanes from [1.1.1]Propellane. Journal of the American Chemical Society, 1992. 114(2): p. 601-620.

8. Martin, T., U. Obst, and J. Rebek, Molecular Assembly and Encapsulation Directed By Hydrogen-Bonding Preferences and the Filling of Space. Science, 1998. 281: p. 1842-1845.

9. Cantrill, S. J., A. R. Pease, and J. F. Stoddart, A molecular meccano kit. Journal of the Chemical Society, Dalton Transactions, 2000: p. 3715-3734.

10. Lehn, J. M., Supramolecular Chemistry—Scope and Perspectives Molecules, Supermolecules, and Molecular Devices. Angewandte Chemie-International Edition in English, 1988. 27(1): p. 89-112.

11. Vogtle, F., et al., Functional dendrimers. Progress in Polymer Science, 2000. 25(7): p. 987-1041.

12. Semlyen, J. A., ed. Large Ring Molecules. 1996, Wiley: New York.

13. Cheng, R. P., S. H. Gellman, and W. F. DeGrado, beta-peptides: From structure to function. Chemical Reviews, 2001. 101(10): p. 3219-3232.

14. Gellman, S. H., Foldamers: A Manifesto. Acc. Chem. Res., 1998. 31: p. 173-180.

15. Seebach, D., et al., Pleated sheets and turns of beta-peptides with proteinogenic side chains. Angewandte Chemie-International Edition, 1999. 38(11): p. 1595-1597.

16. Lazaridis, T., A. Masunov, and F. Gandolfo, Contributions to the binding free energy of ligands to avidin and streptavidin. Proteins, 2002. 47(2): p. 194-208.

17. Wilcox, C. S., Design, Synthesis, and Evaluation of an Efficacious Functional Group Dyad. Methods and Limitations in the Use of NMR for Measuring Host-Guest Interactions, in Frontiers in Supramolecular Organic Chemistry and Photochemistry, H.-J. Schneider and H. Durr, Editors. 1991, VCH Verlagsgesellschaft: New York. p. 123-143.

18. Cram, D. J., The Design of Molecular Hosts, Guests, and Their Complexes (Nobel Lecture). Angewandte Chemie International Edition, 1988. 27(8): p. 1009-1112.

19. Adrian, J. C. and C. S. Wilcox, Orderly Functional Group Dyads. Recognition of Biotin and Adenine Derivatives by a New Synthetic Host. Journal of the American Chemical Society, 1989. 111: p. 8055-8057.

20. Hayashida, O., L. Sebo, and J. Rebek, Molecular Discrimination of N-Protected Amino Acid Esters by a Self-Assembled Cylindrical Capsule: Spectroscopic and Computational Studies. Journal of Organic Chemistry, 2002. 67(24): p. 8291-8298.

21. Dominguez, C., et al., Enantiospecific synthesis of (1S,2S,5R,6S)-2-Aminobicyclo[3.1.0]hexane-2,6-dicarboxylic acid by a modified Corey-Link reaction. Tetrahedron Letters, 1998. 39: p. 9305-9308.

22. Kubik, S., R. S. Meissner, and J. Rebek, Synthesis of Alpha,Alpha-Dialkylated Amino-Acids with Adenine or Thymine Residues—a New Mild and Facile Hydrolysis of Hydantoins. Tetrahedron Letters, 1994. 35(36): p. 6635-6638.

23. Carpino, L. A., 1-Hydroxy-7-azabenzotriazole. An Efficient Peptide Coupling Additive. Journal of the American Chemical Society, 1993. 115: p. 4397-4398.

24. Gisin, B. F. and R. B. Merrifield, Carboxyl-Catalyzed Intramolecular Aminolysis. A Side Reaction in Solid-Phase Peptide Synthesis. Journal of the American Chemical Society, 1972. 94: p. 3102-3106.

25. Pedroso, E., et al., Diketopiperazine Formation in Solid-Phase Peptide-Synthesis Using P-Alkoxybenzyl Ester Resins and Fmoc-Amino Acids. Tetrahedron Letters, 1986. 27(6): p. 743-746.

26. Schafmeister, C. E., L. J. Miercke, and R. M. Stroud, Structure at 2.5 angstroms of a designed peptide that maintains solubility of membrane proteins. Science, 1993. 262: p. 734-738.

27. Cornell, W. D., et al., A 2nd Generation Force-Field for the Simulation of Proteins, Nucleic-Acids, and Organic-Molecules. Journal of the American Chemical Society, 1995. 117(19): p. 5179-5197.

28. MOE, MOE (The Molecular Operating Environment). 2002, Chemical Computing Group Inc.: 1010 Sherbrooke Street West, Suite 910, Montreal, Canada H3A 2R7.

29. Clore, G. M. and A. M. Gronenborn, New methods of structure refinement for macromolecular structure determination by NMR. Proceedings of the National Academy of Sciences of the United States of America, 1998. 95: p. 5891-5898.

30. Schnolzer, M. and S. B. H. Kent, Constructing Proteins by Dovetailing Unprotected Synthetic Peptides—Backbone-Engineered Hiv Protease. Science, 1992. 256(5054): p. 221-225.

31. Pennington, M. W. and M. E. Byrnes, Procedures to Improve Difficult Couplings, in Methods in Molecular Biology: Vol. 35: Peptide Synthesis Protocols, M. W. Pennington and B. M. Dunn, Editors. 1994, Humana Press Inc: Totowa, N.J. p. 1-16.

32. Christie, B. D. and H. Rapoport, Synthesis of Optically Pure Pipecolates from L-Asparagine. Application to the Total Synthesis of (+)-Apovincamine through Amino Acid Decarbonylation and Iminium Ion Cyclization. Journal of Organic Chemistry, 1985. 50: p. 1239-1246.

33. Kamenecka, T. M., et al., Enantioselective approach to 3-substituted prolines. Tetrahedron Letters, 2001. 42: p. 8571-8573.

34. Edward, J. T. and C. Jitrangsri, Stereochemistry of the Bucherer-Bergs and Strecker Reactions of 4-tert-Butylcyclohexanone. Canadian Journal of Chemistry, 1975. 53: p. 3339-3350.

35. Fukuyama, T., C. K. Jow, and M. Cheung, 2-Nitrobenzenesulfonamides and 4-nitrobenzenesulfonamides—Exceptionally versatile means for preparation of secondary-amines and protection of amines. Tetrahedron Letters, 1995. 36(36): p. 6373-6374.

36. Miller, S. C. and T. S. Scanlan, oNBS-SPPS: A New Method for Solid-Phase Peptide Synthesis. Journal of the American Chemical Society, 1998. 120: p. 2690-2691.

37. Bhide, R., et al., A chemoenzymatic synthesis of (+)-castanospermine. Tetrahedron Letters, 1990. 31(34): p. 4827-4830.

38. Porter, E. A., et al., Synthesis and 12-helical secondary structure of beta-peptides containing (2R,3R)-aminoproline. Organic Letters, 2002. 4(19): p. 3317-3319.

39. Pellicciari, R., et al., Enantioselective Synthesis of Naturally Occurring trans-4-Hydroxy-S-Pipecolic Acid-4-Sulfate, A New Potent and Selective NMDA Receptor Agonist. Medicinal Chemistry Research, 1992. 2: p. 491-496.

40. Wessig, P., Stereoselective Synthesis of Novel Chimerical Amino Acids via a Photochemical Key Step. Synlett, 1999: p. 1465-1467.

41. Nelson, R. P., J. M. McEuen, and R. G. Lawton, The (alpha,alpha′) Annelation of Cyclic Ketones. Synthesis of Bicyclo[3.2.1]octane Derivatives. Journal of Organic Chemistry, 1969. 34(5): p. 1225-1229.

What is claimed is:

1. A compound having the formula 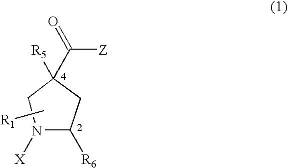
where:
X represents a first amine protecting group;
Y represents a second amine protecting group;
Z represents a weak leaving group;
R₁ represents an H, or a functional group, and can be attached to the molecule at positions 2, 3 or 5;
R₂ represents an H or a functional group;
R₅ represents N₃ or NR₂Y;
R₆ represents a carboxylic acid or a strongly activated ester; and
the stereochemical configuration at positions 2 and 4 and of the carbon bearing R₁ (if R₁ is not H) can be any one of (S,S,S), (S,S,R), (S,R,S), (S,R,R), (R,S,S), (R,S,R), (R,R,S) or (R,R,R).

2. The compound of claim 1, wherein R₅ is N₃.

3. The compound of claim 1, wherein R₅ is NR₂Y.

4. The compound of claim 1, wherein Z is OMe.

5. The compound of claim 1, wherein X is benzylcarbamate.

6. The compound of claim 1, wherein Y is 2-nitrobenzenesulfonamide.

7. The compound of claim 1, wherein Y is 9-fluoroenylmethylcarbamate.

8. The compound of claim 1, wherein X is benzylcarbamate, R₅ is NR₂Y, R₂ is H, Y is 9-fluoroenylmethylcarbamate, Z is —OMe, and R₆ is a carboxylic acid.

9. The compound of claim 1, wherein R₁ is an alkene.

10. The compound of claim 1, wherein R₁ is a protected carboxylate.

11. The compound of claim 1, wherein R₁ is a protected alcohol.

12. The compound of claim 1, wherein R₁ is a protected thiol.

13. A compound having the formula 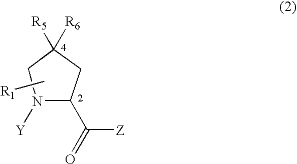
where:
X represents a first amine protecting group;
Y represents a second amine protecting group;
Z represents a weak leaving group;
R₁ represents an H, or a functional group, and can be attached to the molecule at positions 2, 3 or 5;
R₂ represents an H or a functional group;
R₅ represents N₃ or NR₂X;
R₆ represents a carboxylic acid or a strongly activated ester; and
the stereochemical configuration at positions 2 and 4 and of the carbon bearing R₁ (if R₁ is not H) can be any one of (S,S,S), (S,S,R), (S,R,S), (S,R,R), (R,S,S), (R,S,R), (R,R,S) or (R,R,R).

14. The compound of claim 13, wherein R₅ is N₃.

15. The compound of claim 13, wherein R₅ is NR₂X.

16. The compound of claim 13, wherein Z is OMe.

17. The compound of claim 13, wherein X is benzylcarbamate.

18. The compound of claim 13, wherein Y is 2-nitrobenzenesulfonamide.

19. The compound of claim 13, wherein Y is 9-fluoroenylmethylcarbamate.

20. The compound of claim 13, wherein X is benzylcarbamate, R₅ is NR₂X, R₂ is H, Y is 9-fluoroenylmethylcarbamate, Z is —OMe, and R₆ is a carboxylic acid.

21. The compound of claim 13, wherein R₁ is an alkene.

22. The compound of claim 13, wherein R₁ is a protected carboxylate.

23. The compound of claim 13, wherein R₁ is a protected alcohol.

24. The compound of claim 13, wherein R₁ is a protected thiol.

25. A compound having the formula 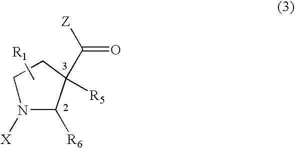
where:
X represents a first amine protecting group;
Y represents a second amine protecting group;
Z represents a weak leaving group;
R₁ represents an H, or a functional group, and can be attached to the molecule at positions 2, 4 or 5;
R₂ represents an H or a functional group;
R₅ represents N₃ or NR₂Y;
R₆ represents a carboxylic acid or a strongly activated ester; and
the stereochemical configuration at positions 2 and 3 and of the carbon bearing R₁ (if R₁ is not H) can be any one of (S,S,S), (S,S,R), (S,R,S), (S,R,R), (R,S,S), (R,S,R), (R,R,S) or (R,R,R).

26. The compound of claim 25, wherein R₅ is N₃.

27. The compound of claim 25, wherein R₅ is NR₂Y.

28. The compound of claim 25, wherein Z is OMe.

29. The compound of claim 25, wherein X is benzylcarbamate.

30. The compound of claim 25, wherein Y is 2-nitrobenzenesulfonamide.

31. The compound of claim 25, wherein Y is 9-fluoroenylmethylcarbamate.

32. The compound of claim 25, wherein X is benzylcarbamate, R₅ is NR₂Y, R₂ is H, Y is 9-fluoroenylmethylcarbamate, Z is —OMe, and R₆ is a carboxylic acid.

33. The compound of claim 25, wherein R₁ is an alkene.

34. The compound of claim 25, wherein R₁ is a protected carboxylate.

35. The compound of claim 25, wherein R₁ is a protected alcohol.

36. The compound of claim 25, wherein R₁ is a protected thiol.

37. A compound having the formula 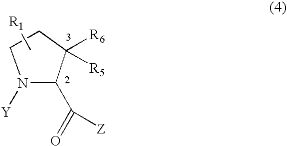
where:
X represents a first amine protecting group;
Y represents a second amine protecting group;
Z represents a weak leaving group;
R₁ represents an H, or a functional group, and can be attached to the molecule at positions 2, 4 or 5;
R₂ represents an H or a functional group;
R₅ represents N₃ or NR₂X;
R₆ represents a carboxylic acid or a strongly activated ester; and
the stereochemical configuration at positions 2 and 3 and of the carbon bearing R₁ (if R₁ is not H) can be any one of (S,S,S), (S,S,R), (S,R,S), (S,R,R), (R,S,S), (R,S,R), (R,R,S) or (R,R,R).

38. The compound of claim 37, wherein R₅ is N₃.

39. The compound of claim 37, wherein R₅ is NR₂X.

40. The compound of claim 37, wherein Z is OMe.

41. The compound of claim 37, wherein X is benzylcarbamate.

42. The compound of claim 37, wherein Y is 2-nitrobenzenesulfonamide.

43. The compound of claim 37, wherein Y is 9-fluoroenylmethylcarbamate.

44. The compound of claim 37, wherein X is benzylcarbamate, R₅ is NR₂X, R₂ is H, Y is 9-fluoroenylmethylcarbamate, Z is —OMe, and R₆ is a carboxylic acid.

45. The compound of claim 37, wherein R₁ is an alkene.

46. The compound of claim 37, wherein R₁ is a protected carboxylate.

47. The compound of claim 37, wherein R₁ is a protected alcohol.

48. The compound of claim 37, wherein R₁ is a protected thiol.

49. A compound having the formula 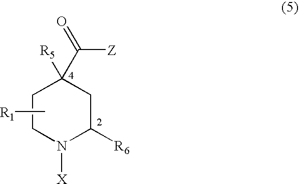
where:
X represents a first amine protecting group;
Y represents a second amine protecting group;
Z represents a weak leaving group;
R₁ represents an H, or a functional group, and can be attached to the molecule at positions 2, 3, 5 or 6;
R₂ represents an H or a functional group;
R₅ represents N₃ or NR₂Y;
R₆ represents a carboxylic acid or a strongly activated ester; and
the stereochemical configuration at positions 2 and 4 and of the carbon bearing R₁ (if R₁ is not H) can be any one of (S,S,S), (S,S,R), (S,R,S), (S,R,R), (R,S,S), (R,S,R), (R,R,S) or (R,R,R).

50. The compound of claim 49, wherein R₅ is N₃.

51. The compound of claim 49, wherein R₅ is NR₂Y.

52. The compound of claim 49, wherein Z is OMe.

53. The compound of claim 49, wherein X is benzylcarbamate.

54. The compound of claim 49, wherein Y is 2-nitrobenzenesulfonamide.

55. The compound of claim 49, wherein Y is 9-fluoroenylmethylcarbamate.

56. The compound of claim 49, wherein X is benzylcarbamate, R₅ is NR₂Y, R₂ is H, Y is 9-fluoroenylmethylcarbamate, Z is —OMe, and R₆ is a carboxylic acid.

57. The compound of claim 49, wherein R₁ is an alkene.

58. The compound of claim 49, wherein R₁ is a protected carboxylate.

59. The compound of claim 49, wherein R₁ is a protected alcohol.

60. The compound of claim 49, wherein R₁ is a protected thiol.

61. A compound having the formula 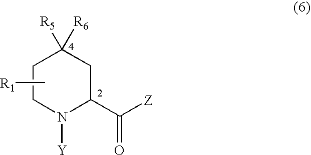
where:
X represents a first amine protecting group;
Y represents a second amine protecting group;
Z represents a weak leaving group;
R₁ represents an H, or a functional group, and can be attached to the molecule at positions 2, 3, 5 or 6;
R₂ represents an H or a functional group;
R₅ represents N₃ or NR₂X;
R₆ represents a carboxylic acid or a strongly activated ester; and
the stereochemical configuration at positions 2 and 4 and of the carbon bearing R₁ (if R₁ is not H) can be any one of (S,S,S), (S,S,R), (S,R,S), (S,R,R), (R,S,S), (R,S,R), (R,R,S) or (R,R,R).

62. The compound of claim 61, wherein R₅ is N₃.

63. The compound of claim 61, wherein R₅ is NR₂X.

64. The compound of claim 61, wherein Z is OMe.

65. The compound of claim 61, wherein X is benzylcarbamate.

66. The compound of claim 61, wherein Y is 2-nitrobenzenesulfonamide.

67. The compound of claim 61, wherein Y is 9-fluoroenylmethylcarbamate.

68. The compound of claim 61, wherein X is benzylcarbamate, R₅ is NR₂X, R₂ is H, Y is 9-fluoroenylmethylcarbamate, Z is —OMe, and R₆ is a carboxylic acid.

69. The compound of claim 61, wherein R₁ is an alkene.

70. The compound of claim 61, wherein R₁ is a protected carboxylate.

71. The compound of claim 61, wherein R₁ is a protected alcohol.

72. The compound of claim 61, wherein R₁ is a protected thiol.

73. A compound having the formula 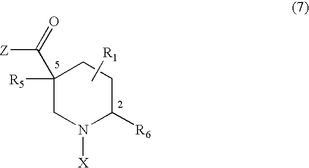
where:
X represents a first amine protecting group;
Y represents a second amine protecting group;
Z represents a weak leaving group;
R₁ represents an H, or a functional group, and can be attached to the molecule at positions 2, 3, 4 or 6;
R₂ represents an H or a functional group;
R₅ represents N₃ or NR₂Y;
R₆ represents a carboxylic acid or a strongly activated ester; and
the stereochemical configuration at positions 2 and 5 and of the carbon bearing R₁ (if R₁ is not H) can be any one of (S,S,S), (S,S,R), (S,R,S), (S,R,R), (R,S,S), (R,S,R), (R,R,S) or (R,R,R).

74. The compound of claim 73, wherein R₅ is N₃.

75. The compound of claim 73, wherein R₅ is NR₂Y.

76. The compound of claim 73, wherein Z is OMe.

77. The compound of claim 73, wherein X is benzylcarbamate.

78. The compound of claim 73, wherein Y is 2-nitrobenzenesulfonamide.

79. The compound of claim 73, wherein Y is 9-fluoroenylmethylcarbamate.

80. The compound of claim 73, wherein X is benzylcarbamate, R₅ is NR₂Y, R₂ is H, Y is 9-fluoroenylmethylcarbamate, Z is —OMe, and R₆ is a carboxylic acid.

81. The compound of claim 73, wherein R₁ is an alkene.

82. The compound of claim 73, wherein R₁ is a protected carboxylate.

83. The compound of claim 73, wherein R₁ is a protected alcohol.

84. The compound of claim 73, wherein R₁ is a protected thiol.

85. A compound having the formula 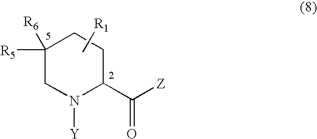
where:
X represents a first amine protecting group;
Y represents a second amine protecting group;
Z represents a weak leaving group;
R₁ represents an H, or a functional group, and can be attached to the molecule at positions 2, 3, 5 or 6;
R₂ represents an H or a functional group;
R₅ represents N₃ or NR₂X;
R₆ represents a carboxylic acid or a strongly activated ester; and
the stereochemical configuration at positions 2 and 5 and of the carbon bearing R₁ (if R₁ is not H) can be any one of (S,S,S), (S,S,R), (S,R,S), (S,R,R), (R,S,S), (R,S,R), (R,R,S) or (R,R,R).

86. The compound of claim 85, wherein R₅ is N₃.

87. The compound of claim 85, wherein R₅ is NR₂X.

88. The compound of claim 85, wherein Z is OMe.

89. The compound of claim 85, wherein X is benzylcarbamate.

90. The compound of claim 85, wherein Y is 2-nitrobenzenesulfonamide.

91. The compound of claim 85, wherein Y is 9-fluoroenylmethylcarbamate.

92. The compound of claim 85, wherein X is benzylcarbamate, R₅ is NR₂X, R₂ is H, Y is 9-fluoroenylmethylcarbamate, Z is —OMe, and R₆ is a carboxylic acid.

93. The compound of claim 85, wherein R₁ is an alkene.

94. The compound of claim 85, wherein R₁ is a protected carboxylate.

95. The compound of claim 85, wherein R₁ is a protected alcohol.

96. The compound of claim 85, wherein R₁ is a protected thiol.

97. A compound having the formula 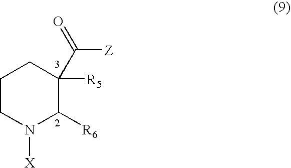
where:
X represents a first amine protecting group;
Y represents a second amine protecting group;
Z represents a weak leaving group;
R₁ represents an H, or a functional group, and can be attached to the molecule at positions 2, 4, 5 or 6;
R₂ represents an H or a functional group;
R₅ represents N₃ or NR₂Y;
R₆ represents a carboxylic acid or a strongly activated ester; and
the stereochemical configuration at positions 2 and 3 and of the carbon bearing R₁ (if R₁ is not H) can be any one of (S,S,S), (S,S,R), (S,R,S), (S,R,R), (R,S,S), (R,S,R), (R,R,S) or (R,R,R).

98. The compound of claim 97, wherein R₅ is N₃.

99. The compound of claim 97, wherein R₅ is NR₂Y.

100. The compound of claim 97, wherein Z is OMe.

101. The compound of claim 97, wherein X is benzylcarbamate.

102. The compound of claim 97, wherein Y is 2-nitrobenzenesulfonamide.

103. The compound of claim 97, wherein Y is 9-fluoroenylmethylcarbamate.

104. The compound of claim 97, wherein X is benzylcarbamate, R₅ is NR₂Y, R₂ is H, Y is 9-fluoroenylmethylcarbamate, Z is —OMe, and R₆ is a carboxylic acid.

105. The compound of claim 97, wherein R₁ is an alkene.

106. The compound of claim 97, wherein R₁ is a protected carboxylate.

107. The compound of claim 97, wherein R₁ is a protected alcohol.

108. The compound of claim 97, wherein R₁ is a protected thiol.

109. A compound having the formula 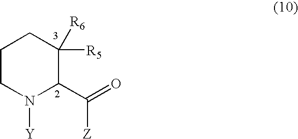
where:
X represents a first amine protecting group;
Y represents a second amine protecting group;
Z represents a weak leaving group;
R₁ represents an H, or a functional group, and can be attached to the molecule at positions 2, 4, 5 or 6;
R₂ represents an H or a functional group;
R₅ represents N₃ or NR₂X;
R₆ represents a carboxylic acid or a strongly activated ester; and
the stereochemical configuration at positions 2 and 3 and of the carbon bearing R₁ (if R₁ is not H) can be any one of (S,S,S), (S,S,R), (S,R,S), (S,R,R), (R,S,S), (R,S,R), (R,R,S) or (R,R,R).

110. The compound of claim 109, wherein R₅ is N₃.

111. The compound of claim 109, wherein R₅ is NR₂X.

112. The compound of claim 109, wherein Z is OMe.

113. The compound of claim 109, wherein X is benzylcarbamate.

114. The compound of claim 109, wherein Y is 2-nitrobenzenesulfonamide.

115. The compound of claim 109, wherein Y is 9-fluoroenylmethylcarbamate.

116. The compound of claim 109, wherein X is benzylcarbamate, R₅ is NR₂X, R₂ is H, Y is 9-fluoroenylmethylcarbamate, Z is —OMe, and R₆ is a carboxylic acid.

117. The compound of claim 109, wherein R₁ is an alkene.

118. The compound of claim 109, wherein R₁ is a protected carboxylate.

119. The compound of claim 109, wherein R₁ is a protected alcohol.

120. The compound of claim 109, wherein R₁ is a protected thiol.

121. A compound having the formula 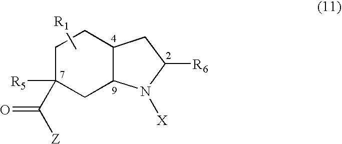
where:
X represents a first amine protecting group;
Y represents a second amine protecting group;
Z represents a weak leaving group;
R₁ represents an H, or a functional group, and can be attached to the molecule at positions 2, 3, 4, 5, 6, 8 or 9;
R₂ represents an H or a functional group;
R₅ represents N₃ or NR₂Y;
R₆ represents a carboxylic acid or a strongly activated ester; and
the stereochemical configuration at positions 2, 4, 7, 9 and of the carbon bearing R₁ (if R₁ is not H) can be any of the 32 combinations of (R) and (S).

122. The compound of claim 121, wherein R₅ is N₃.

123. The compound of claim 121, wherein R₅ is NR₂Y.

124. The compound of claim 121, wherein Z is OMe.

125. The compound of claim 121, wherein X is benzylcarbamate.

126. The compound of claim 121, wherein Y is 2-nitrobenzenesulfonamide.

127. The compound of claim 121, wherein Y is 9-fluoroenylmethylcarbamate.

128. The compound of claim 121, wherein X is benzylcarbamate, R₅ is NR₂Y, R₂ is H, Y is 9-fluoroenylmethylcarbamate, Z is —OMe, and R₆ is a carboxylic acid.

129. The compound of claim 121, wherein R₁ is an alkene.

130. The compound of claim 121, wherein R₁ is a protected carboxylate.

131. The compound of claim 121, wherein R₁ is a protected alcohol.

132. The compound of claim 121, wherein R₁ is a protected thiol.

133. A compound having the formula 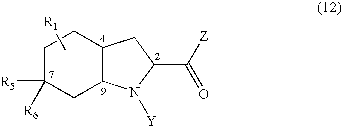
where:
X represents a first amine protecting group;
Y represents a second amine protecting group;
Z represents a weak leaving group;
R₁ represents an H, or a functional group, and can be attached to the molecule at positions 2, 3, 4, 5, 6, 8 or 9;
R₂ represents an H or a functional group;
R₅ represents N₃ or NR₂X;
R₆ represents a carboxylic acid or a strongly activated ester; and
the stereochemical configuration at positions 2, 4, 7, 9 and of the carbon bearing R₁ (if R₁ is not H) can be any of the 32 combinations of (R) and (S).

134. The compound of claim 133, wherein R₅ is N₃.

135. The compound of claim 133, wherein R₅ is NR₂X.

136. The compound of claim 133, wherein Z is OMe.

137. The compound of claim 133, wherein X is benzylcarbamate.

138. The compound of claim 133, wherein Y is 2-nitrobenzenesulfonamide.

139. The compound of claim 133, wherein Y is 9-fluoroenylmethylcarbamate.

140. The compound of claim 133, wherein X is benzylcarbamate, R₅ is NR₂X, R₂ is H, Y is 9-fluoroenylmethylcarbamate, Z is —OMe, and R₆ is a carboxylic acid.

141. The compound of claim 133, wherein R₁ is an alkene.

142. The compound of claim 133, wherein R₁ is a protected carboxylate.

143. The compound of claim 133, wherein R₁ is a protected alcohol.

144. The compound of claim 133, wherein R₁ is a protected thiol.

145. A compound having the formula 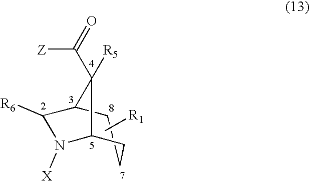
where:
X represents a first amine protecting group;
Y represents a second amine protecting group;
Z represents a weak leaving group;
R₁ represents an H, or a functional group, and can be attached to the molecule at positions 2, 3, 5, 6, 7 or 8;
R₂ represents an H or a functional group;
R₅ represents N₃ or NR₂Y;
R₆ represents a carboxylic acid or a strongly activated ester; and
the stereochemical configuration at the positions 2, 3, 4 and 5, and of the carbon bearing R₁ (if R₁ is not H) can be any of the 32 combinations of (R) and (S).

146. The compound of claim 145, wherein R₅ is N₃.

147. The compound of claim 145, wherein R₅ is NR₂Y.

148. The compound of claim 145, wherein Z is OMe.

149. The compound of claim 145, wherein X is benzylcarbamate.

150. The compound of claim 145, wherein Y is 2-nitrobenzenesulfonamide.

151. The compound of claim 145, wherein Y is 9-fluoroenylmethylcarbamate.

152. The compound of claim 145, wherein X is benzylcarbamate, R₅ is NR₂Y, R₂is H, Y is 9-fluoroenylmethylcarbamate, Z is —OMe, and R₆ is a carboxylic acid.

153. The compound of claim 145, wherein R₁ is an alkene.

154. The compound of claim 145, wherein R₁ is a protected carboxylate.

155. The compound of claim 145, wherein R₁ is a protected alcohol.

156. The compound of claim 145, wherein R₁ is a protected thiol.

157. A compound having the formula 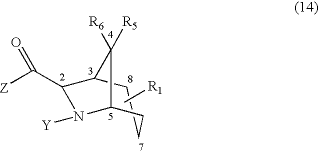
where:
X represents a first amine protecting group;
Y represents a second amine protecting group;
Z represents a weak leaving group;
R₁ represents an H, or a functional group, and can be attached to the molecule at positions 2, 3, 5, 6, 7 or 8;
R₂ represents an H or a functional group;
R₅ represents N₃ or NR₂X;
R₆ represents a carboxylic acid or a strongly activated ester; and
the stereochemical configuration at the positions 2, 3, 4 and 5, and of the carbon bearing R₁ (if R₁ is not H) can be any of the 32 combinations of (R) and (S).

158. The compound of claim 157, wherein R₅ is N₃.

159. The compound of claim 157, wherein R₅ is NR₂X.

160. The compound of claim 157, wherein Z is OMe.

161. The compound of claim 157, wherein X is benzylcarbamate.

162. The compound of claim 157, wherein Y is 2-nitrobenzenesulfonamide.

163. The compound of claim 157, wherein Y is 9-fluoroenylmethylcarbamate.

164. The compound of claim 157, wherein X is benzylcarbamate, R₅ is NR₂X, R₂ is H, Y is 9-fluoroenylmethylcarbamate, Z is —OMe, and R₆ is a carboxylic acid.

165. The compound of claim 157, wherein R₁ is an alkene.

166. The compound of claim 157, wherein R₁ is a protected carboxylate.

167. The compound of claim 157, wherein R₁ is a protected alcohol.

168. The compound of claim 157, wherein R₁ is a protected thiol.

169. A method of synthesizing bis peptides comprising the steps of:
1) providing a solid support;
2) activating a first bis amino acid or naturally occurring amino acid;
3) attaching the bis amino acid or naturally occurring amino acid to the support;
4) removing the leading edge amine protecting group if a bis amino acid is used, or the amine protecting group if a naturally occurring amino acid is used;
5) activating and attaching a next bis amino acid or a next naturally occurring amino acid to the leading edge amine of the bis amino acid or amine of the naturally occurring amino acid; and
6) repeating steps 4 and 5 as necessary to achieve the desired chain length;
7) detaching the synthesized bis peptide from the support; and
8) isolating the synthesized bis peptide,
where the bis peptide synthesized in the above manner has at least two contiguous bis amino acids, and a rigidification step is carried out either after step 4 or after detachment of the bis peptide from the solid support.

170. The method of claim 169, further comprising the step of modifying or adding a functional group, after step 5.

171. A method of synthesizing bis peptides comprising the steps of:
1) providing a bis-amino acid or bis-peptide fragment containing a mixture of bis-amino acid and naturally occurring amino acid with an unprotected leading edge amine and a protected trailing edge carboxylic acid;
2) providing a bis-s or bis-peptide fragment containing a mixture of bis-amino acid and naturally occurring amino acids with a protected leading edge amine and an activated ester;
3) coupling the two fragments in solution;
4) isolating the synthesized bis-peptide;
5) removing the leading edge amine protecting group or the trailing end carboxylic acid protecting group; and
6) repeating steps 1,2,3,4 to achieve the desired chain length;
where the bis peptide synthesized in the above manner has at least two contiguous bis amino acids, and a rigidification step is carried out either after step 3 or after detachment of the bis peptide from the solid support.

172. The method of claim 171, further comprising the step of modifiying or adding a functional group, after step 3.

173. A compound having the formula 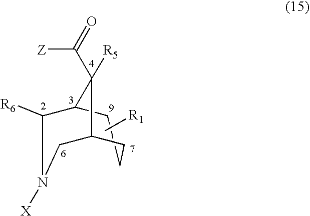
where:
X represents a first amine protecting group;
Y represents a second amine protecting group;
Z represents a weak leaving group;
R₁ represents an H, or a functional group, and can be attached to the molecule at positions 2, 3, 5, 6, 7, 8 or 9;
R₂ represents an H or a functional group;
R₅ represents N₃ or NR₂Y;
R₆ represents a carboxylic acid or a strongly activated ester; and
the stereochemical configuration at the positions 2, 3, 4 and 5, and of the carbon bearing R₁ (if R₁ is not H) can be any of the 32 combinations of (R) and (S).

174. The compound of claim 173, wherein R₅ is N₃.

175. The compound of claim 173, wherein R₅ is NR₂Y.

176. The compound of claim 173, wherein Z is OMe.

177. The compound of claim 173, wherein X is benzylcarbamate.

178. The compound of claim 173, wherein Y is 2-nitrobenzenesulfonamide.

179. The compound of claim 173, wherein Y is 9-fluoroenylmethylcarbamate.

180. The compound of claim 173, wherein X is benzylcarbamate, R₅ is NR₂Y, R₂ is H, Y is 9-fluoroenylmethylcarbamate, Z is —OMe, and R₆ is a carboxylic acid.

181. The compound of claim 173, wherein R₁ is an alkene.

182. The compound of claim 173, wherein R₁ is a protected carboxylate.

183. The compound of claim 173, wherein R₁ is a protected alcohol.

184. The compound of claim 173, wherein R₁ is a protected thiol.

185. A compound having the formula 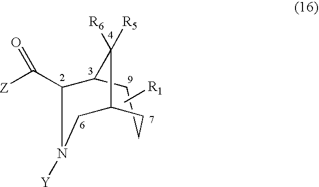
where:
X represents a first amine protecting group;
Y represents a second amine protecting group;
Z represents a weak leaving group;
R₁ represents an H, or a functional group, and can be attached to the molecule at positions 2, 3, 5, 6, 7, 8 or 9;
R₂ represents an H or a functional group;
R₅ represents N₃ or NR₂X;
R₆ represents a carboxylic acid or a strongly activated ester; and
the stereochemical configuration at the positions 2, 3, 4 and 5, and of the carbon bearing R₁ (if R₁ is not H) can be any of the 32 combinations of (R) and (S).

186. The compound of claim 185, wherein R₅ is N₃.

187. The compound of claim 185, wherein R₅ is NR₂X.

188. The compound of claim 185, wherein Z is OMe.

189. The compound of claim 185, wherein X is benzylcarbamate.

190. The compound of claim 185, wherein Y is 2-nitrobenzenesulfonamide.

191. The compound of claim 185, wherein Y is 9-fluoroenylmethylcarbamate.

192. The compound of claim 185, wherein X is benzylcarbamate, R₅ is NR₂X, R₂ is H, Y is 9-fluoroenylmethylcarbamate, Z is —OMe, and R₆ is a carboxylic acid.

193. The compound of claim 185, wherein R₁ is an alkene.

194. The compound of claim 185, wherein R₁ is a protected carboxylate.

195. The compound of claim 185, wherein R₁ is a protected alcohol.

196. The compound of claim 185, wherein R₁ is a protected thiol.

197. A compound having the formula 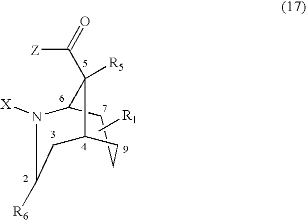
where:
X represents a first amine protecting group;
Y represents a second amine protecting group;
Z represents a weak leaving group;
R₁ represents an H, or a functional group, and can be attached to the molecule at positions 2, 3, 4, 6, 7, 8 or 9;
R₂ represents an H or a functional group;
R₅ represents N₃ or NR₂Y;
R₆ represents a carboxylic acid or a strongly activated ester; and
the stereochemical configuration at the positions 2, 4, 5 and 6, and of the carbon bearing R₁ (if R₁ is not H) can be any of the 32 combinations of (R) and (S).

198. The compound of claim 197, wherein R₅ is N₃.

199. The compound of claim 197, wherein R₅ is NR₂Y.

200. The compound of claim 197, wherein Z is OMe.

201. The compound of claim 197, wherein X is benzylcarbamate.

202. The compound of claim 197, wherein Y is 2-nitrobenzenesulfonamide.

203. The compound of claim 197, wherein Y is 9-fluoroenylmethylcarbamate.

204. The compound of claim 197, wherein X is benzylcarbamate, R₅ is NR₂Y, R₂ is H, Y is 9-fluoroenylmethylcarbamate, Z is —OMe, and R₆ is a carboxylic acid.

205. The compound of claim 197, wherein R₁ is an alkene.

206. The compound of claim 197, wherein R₁ is a protected carboxylate.

207. The compound of claim 197, wherein R₁ is a protected alcohol.

208. The compound of claim 197, wherein R₁ is a protected thiol.

209. A compound having the formula 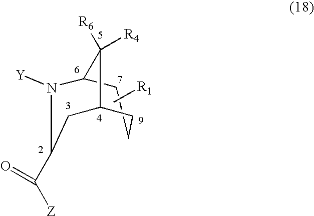
where:
X represents a first amine protecting group;
Y represents a second amine protecting group;
Z represents a weak leaving group;
R₁ represents an H, or a functional group, and can be attached to the molecule at positions 2, 3, 4, 6, 7, 8 or 9;
R₂ represents an H or a functional group;
R₅ represents N₃ or NR₂X;
R₆ represents a carboxylic acid or a strongly activated ester; and
the stereochemical configuration at the positions 2, 4, 5 and 6, and of the carbon bearing R₁ (if R₁ is not H) can be any of the 32 combinations of (R) and (S).

210. The compound of claim 209, wherein R₅ is N₃.

211. The compound of claim 209, wherein R₅ is NR₂X.

212. The compound of claim 209, wherein Z is OMe.

213. The compound of claim 209, wherein X is benzylcarbamate.

214. The compound of claim 209, wherein Y is 2-nitrobenzenesulfonamide.

215. The compound of claim 209, wherein Y is 9-fluoroenylmethylcarbamate.

216. The compound of claim 209, wherein X is benzylcarbamate, R₅ is NR₂X, R₂ is H, Y is 9-fluoroenylmethylcarbamate, Z is —OMe, and R₆ is a carboxylic acid.

217. The compound of claim 209, wherein R₁ is an alkene.

218. The compound of claim 209, wherein R₁ is a protected carboxylate.

219. The compound of claim 209, wherein R₁ is a protected alcohol.

220. The compound of claim 209, wherein R₁ is a protected thiol.

221. A compound having the formula 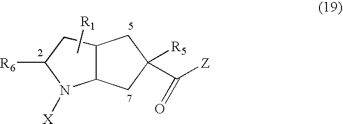
where:
X represents a first amine protecting group;
Y represents a second amine protecting group;
Z represents a weak leaving group;
R₁ represents an H, or a functional group, and can be attached to the molecule at positions 2, 3, 4, 5, 7 or 8;
R₂ represents an H or a functional group;
R₅ represents N₃ or NR₂Y;
R₆ represents a carboxylic acid or a strongly activated ester; and
the stereochemical configuration at the positions 2, 4, 6 and 8, and of the carbon bearing R₁ (if R₁ is not H) can be any of the 32 combinations of (R) and (S).

222. The compound of claim 221, wherein R₅ is N₃.

223. The compound of claim 221, wherein R₅ is NR₂Y.

224. The compound of claim 221, wherein Z is OMe.

225. The compound of claim 221, wherein X is benzylcarbamate.

226. The compound of claim 221, wherein Y is 2-nitrobenzenesulfonamide.

227. The compound of claim 221, wherein Y is 9-fluoroenylmethylcarbamate.

228. The compound of claim 221, wherein X is benzylcarbamate, R₅ is NR₂Y, R₂ is H, Y is 9-fluoroenylmethylcarbamate, Z is —OMe, and R₆ is a carboxylic acid.

229. The compound of claim 221, wherein R₁ is an alkene.

230. The compound of claim 221, wherein R₁ is a protected carboxylate.

231. The compound of claim 221, wherein R₁ is a protected alcohol.

232. The compound of claim 221, wherein R₁ is a protected thiol.

233. A compound having the formula 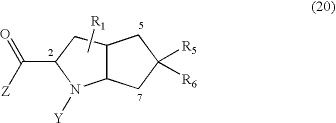
where:
X represents a first amine protecting group;
Y represents a second amine protecting group;
Z represents a weak leaving group;
R₁ represents an H, or a functional group, and can be attached to the molecule at positions 2, 3, 4, 5, 7 or 8;
R₂ represents an H or a functional group;
R₅ represents N₃ or NR₂X;
R₆ represents a carboxylic acid or a strongly activated ester; and
the stereochemical configuration at the positions 2, 4, 6 and 8, and of the carbon bearing R₁ (if R₁ is not H) can be any of the 32 combinations of (R) and (S).

234. The compound of claim 233, wherein R₅ is N₃.

235. The compound of claim 233, wherein R₅ is NR₂X.

236. The compound of claim 233, wherein Z is OMe.

237. The compound of claim 233, wherein X is benzylcarbamate.

238. The compound of claim 233, wherein Y is 2-nitrobenzenesulfonamide.

239. The compound of claim 233, wherein Y is 9-fluoroenylmethylcarbamate.

240. The compound of claim 233, wherein X is benzylcarbamate, R₅ is NR₂X, R₂ is H, Y is 9-fluoroenylmethylcarbamate, Z is —OMe, and R₆ is a carboxylic acid.

241. The compound of claim 233, wherein R₁ is an alkene.

242. The compound of claim 233, wherein R₁ is a protected carboxylate.

243. The compound of claim 233, wherein R₁ is a protected alcohol.

244. The compound of claim 233, wherein R₁ is a protected thiol.

245. A compound having the formula 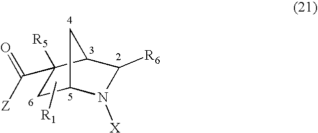
where:
X represents a first amine protecting group;
Y represents a second amine protecting group;
Z represents a weak leaving group;
R₁ represents an H, or a functional group, and can be attached to the molecule at positions 2, 3, 4, 5 or 6;
R₂ represents an H or a functional group;
R₅ represents N₃ or NR₂Y;
R₆ represents a carboxylic acid or a strongly activated ester; and
the stereochemical configuration at the positions 2, 3, 5 and 7, and of the carbon bearing R₁ (if R₁ is not H) can be any of the 32 combinations of (R) and (S).

246. The compound of claim 245, wherein R₅ is N₃.

247. The compound of claim 245, wherein R₅ is NR₂Y.

248. The compound of claim 245, wherein Z is OMe.

249. The compound of claim 245, wherein X is benzylcarbamate.

250. The compound of claim 245, wherein Y is 2-nitrobenzenesulfonamide.

251. The compound of claim 245, wherein Y is 9-fluoroenylmethylcarbamate.

252. The compound of claim 245, wherein X is benzylcarbamate, R₅ is NR₂Y, R₂ is H, Y is 9-fluoroenylmethylcarbamate, Z is —OMe, and R₆ is a carboxylic acid.

253. The compound of claim 245, wherein R₁ is an alkene.

254. The compound of claim 245, wherein R₁ is a protected carboxylate.

255. The compound of claim 245, wherein R₁ is a protected alcohol.

256. The compound of claim 245, wherein R₁ is a protected thiol.

257. A compound having the formula 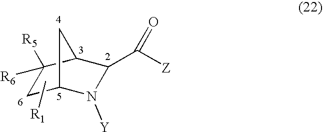
where:
X represents a first amine protecting group;
Y represents a second amine protecting group;
Z represents a weak leaving group;
R₁ represents an H, or a functional group, and can be attached to the molecule at positions 2, 3, 4, 5 or 6;
R₂ represents an H or a functional group;
R₅ represents N₃ or NR₂X;
R₆ represents a carboxylic acid or a strongly activated ester; and
the stereochemical configuration at the positions 2, 3, 5 and 7, and of the carbon bearing R₁ (if R₁ is not H) can be any of the 32 combinations of (R) and (S).

258. The compound of claim 257, wherein R₅ is N₃.

259. The compound of claim 257, wherein R₅ is NR₂X.

260. The compound of claim 257, wherein Z is OMe.

261. The compound of claim 257, wherein X is benzylcarbamate.

262. The compound of claim 257, wherein Y is 2-nitrobenzenesulfonamide.

263. The compound of claim 257, wherein Y is 9-fluoroenylmethylcarbamate.

264. The compound of claim 257, wherein X is benzylcarbamate, R₅ is NR₂X, R₂ is H, Y is 9-fluoroenylmethylcarbamate, Z is —OMe, and R₆ is a carboxylic acid.

265. The compound of claim 257, wherein R₁ is an alkene.

266. The compound of claim 257, wherein R₁ is a protected carboxylate.

267. The compound of claim 257, wherein R₁ is a protected alcohol.

268. The compound of claim 257, wherein R₁ is a protected thiol.

269. A compound having the formula 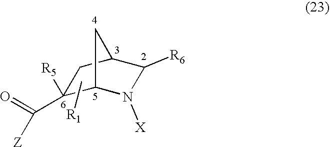
where:
X represents a first amine protecting group;
Y represents a second amine protecting group;
Z represents a weak leaving group;
R₁ represents an H, or a functional group, and can be attached to the molecule at positions 2, 3, 4, 5 or 7;
R₂ represents an H or a functional group;
R₅ represents N₃ or NR₂Y;
R₆ represents a carboxylic acid or a strongly activated ester; and
the stereochemical configuration at the positions 2, 3, 5 and 6, and of the carbon bearing R₁ (if R₁ is not H) can be any of the 32 combinations of (R) and (S).

270. The compound of claim 269, wherein R₅ is N₃.

271. The compound of claim 269, wherein R₅ is NR₂Y.

272. The compound of claim 269, wherein Z is OMe.

273. The compound of claim 269, wherein X is benzylcarbamate.

274. The compound of claim 269, wherein Y is 2-nitrobenzenesulfonamide.

275. The compound of claim 269, wherein Y is 9-fluoroenylmethylcarbamate.

276. The compound of claim 269, wherein X is benzylcarbamate, R₅ is NR₂Y, R₂ is H, Y is 9-fluoroenylmethylcarbamate, Z is —OMe, and R₆ is a carboxylic acid.

277. The compound of claim 269, wherein R₁ is an alkene.

278. The compound of claim 269, wherein R₁ is a protected carboxylate.

279. The compound of claim 269, wherein R₁ is a protected alcohol.

280. The compound of claim 269, wherein R₁ is a protected thiol.

281. A compound having the formula 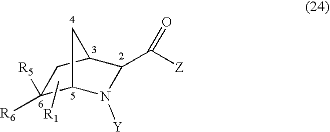
where:
X represents a first amine protecting group;
Y represents a second amine protecting group;
Z represents a weak leaving group;
R₁ represents an H, or a functional group, and can be attached to the molecule at positions 2, 3, 4, 5 or 7;
R₂ represents an H or a functional group;
R₅ represents N₃ or NR₂X;
R₆ represents a carboxylic acid or a strongly activated ester; and
the stereochemical configuration at the positions 2, 3, 5 and 6, and of the carbon bearing R₁ (if R₁ is not H) can be any of the 32 combinations of (R) and (S).

282. The compound of claim 281, wherein R₅ is N₃.

283. The compound of claim 281, wherein R₅ is NR₂X.

284. The compound of claim 281, wherein Z is OMe.

285. The compound of claim 281, wherein X is benzylcarbamate.

286. The compound of claim 281, wherein Y is 2-nitrobenzenesulfonamide.

287. The compound of claim 281, wherein Y is 9-fluoroenylmethylcarbamate.

288. The compound of claim 281, wherein X is benzylcarbamate, R₅ is NR₂X, R₂ is H, Y is 9-fluoroenylmethylcarbamate, Z is —OMe, and R₆ is a carboxylic acid.

289. The compound of claim 281, wherein R₁ is an alkene.

290. The compound of claim 281, wherein R₁ is a protected carboxylate.

291. The compound of claim 281, wherein R₁ is a protected alcohol.

292. The compound of claim 281, wherein R₁ is a protected thiol.

293. A synthesized bis peptide made by the method of claim 169, where the number of amino acids in the peptide, whether naturally occurring or bis amino acids, is less than 500.

294. A synthesized bis peptide made by the method of claim 171, where the number of amino acids in the peptide, whether naturally occurring or bis amino acids, is less than 500.
